(19) United States
(12) Patent Application Publication
(10) Pub. No.: US 2015/0246087 A1
(43) Pub. Date: Sep. 3, 2015

(54) EXTRACTS AND THERAPEUTIC USES THEREOF

(71) Applicants: Zadik Hazan, Zikron Yaakov (IL); Andre C.B. Lucassen, Rehovot (IL)

(72) Inventors: Zadik Hazan, Zikron Yaakov (IL); Andre C.B. Lucassen, Rehovot (IL)

(73) Assignee: REGENERA PHARMA LTD., Rehovot (IL)

(21) Appl. No.: 14/407,049

(22) PCT Filed: Jun. 11, 2012
(86) PCT No.: PCT/IL2012/000232

§ 371 Date: May 12, 2015

(51) Int. Cl. A61K 36/14

(52) U.S. Cl. CPC A61K 36/14; A61K 2236/39

(57) ABSTRACT

The invention relates to compositions and formulations comprising isolated fractions derived from Cupressaceae plant material. More particularly, the invention relates to pharmaceutical compositions comprising an isolated fraction of Cupressaceae in a carrier and use thereof for treating fibrotic conditions and neurodegenerative disorders.

### FIELD OF THE INVENTION

The invention relates to extracts of Cupressaceae products, including resins, and therapeutic uses thereof. More particularly, the invention provides isolated fractions of Cupressaceae and compositions comprising such fractions as the pharmaceutically active ingredient.

### BACKGROUND OF THE INVENTION

Essential oils and extracts derived from gymnosperms of the family Cupressaceae (cypress trees) have been disclosed to have a wide range of therapeutic activities, such as that directed against various infectious agents. For example, anti-fungal activity of essential oils from Thuja sutchuenensis (Lei et al., Nat Prod Commun. 2010 October; 5(10):1673-6); antimicrobial activity of essential oils from cabreuva (Myrocarpus fastigiatus), cedarwood (Juniperus ashei), and juniper (Juniperus communis L. and Juniperus phoenicea) (Wanner et al., Nat Prod Commun. 2010 September; 5(9):1359-64; Ennajar et al., J Food Sci. 2009 September; 74(7):M364-71); and anti-HIV-1 activity of ethanol extracts from Cupressus sempervirens, have been reported (Iran. J. Basic Med. Sc. Vol. 12 (3-4), 133-39, 2009).

KR 20030033722 discloses a natural insecticidal composition containing an oil extracted from Cupressus sempervirens.

JP 8169839 discloses a tar- and acid-free resin oil extract from Chamacyparis Spach and Thujopsis dolabrata, said extract having anti-bacterial and anti-mite activities, and additional beneficial effects such as wound healing, promoting epithelialization, and improving blood circulation.

WO 98/051319 discloses a pharmaceutical composition comprising a mixture of Angelica oil from Angelica archangelica L., Umbelliferae and Red Cedar oil from Juniperus virginiana L., Cupressaceae, and use thereof for treating and/or preventing infectious and immune diseases, in particular that caused by HIV, as well as neoplasic pathologies.

Ethanolic and aqueous extracts derived from berries of Chinese juniper (Juniperus chinensis L.) have been disclosed to have hypoglycemic and hypolipidemic properties, respectively (Ju et al., J Ethnopharmacol. 2008 Jan. 4; 115(1):110-5).

WO 99/07398 discloses that extracts and powders isolated from Cypraesus semprevirens and all species of cypress are effective for preventing and treating hypercholesterolemia and lipid disturbances.

Oils from Chamaecyparis obtusa have been disclosed to promote hair growth in animals, and certain components of these oils reportedly induce expression of VEGF a positive regulator of hair growth (Lee et al., Fitoterapia. 2010 January; 81(1):17-24).

WO 94/23732 discloses a therapeutic preparation from Cypress trees, such as an oil or extract, and use thereof for treating psoriasis and other itching diseases, radiation or radiotherapy skin damage, leprosy, cancer and leukemia.

CN 101327231 discloses a medicinal herb mixture containing cypress resin crystal as one of several botanical ingredients for the treatment of psoriasis.

WO 2008/140200 discloses an external composition for skin, comprising sulfur and alum, and optionally further comprising a specified proportion of a plant extract or plant oil from Cupressaceae, and use thereof for treating psoriasis, blistery tinea, eczema, itch, shingles, chronic pruritus, pustulosis palmaris et plantaris, fungal tinea pedis and malignant intumescence.

Methanolic extracts of certain Juniperus species have been disclosed to exhibit anti-inflammatory and anti-nociceptive activities (Akkol et al., J Ethnopharmacol. 2009 Sep. 7; 125(2):330-6).

WO 2002/047707 discloses a method for inhibiting COX-2 activity, comprising administering a composition comprising an organic extract of a non-edible plant, wherein the plant may be from the order Coniferales; and use of said extract for treating or preventing COX-2 mediated inflammation or an inflammation-associated disorder, inter alia arthritis, cancer, or a central nervous system disorder such as Alzheimer's Disease. According to the disclosure, the extract may be obtained using hydrocarbon solvents, ether solvents, chlorinated solvents, acetone, ethyl acetate, butanol, ethanol, methanol, isopropyl alcohol and mixtures thereof.

Systematic investigations into the constituents of different Cupressaceae extracts have been reported (see for example, Enzell, Acta Chem. Sc. 1961, 15, 1303-12).

Various terpene and terpenoid compounds have been extracted from leaves, bark, wood, roots and twigs of Cupressaceae family members. Essential oils derived from such plant parts have been disclosed to consist mainly of monoterpenes and sesquiterpenes such as α-pinene, β-pinene, sabinene, limonene, bisabolol, cedrene, farnesol, longifolene and cadinene (see for example, Ennajar et al., J Food Sci. 2009 September; 74(7):M364-71). Diterpene derivatives isolated from non-volatile extracts include for example sempervirol, totarol, ferruginol, manool, and torusolol (Piovetti et al. Phytochem. 19, 1980, 2772-3). Triterpenoids termed chamaecydines were disclosed as having been isolated from Chamaecyparis and Cryptomeria (Otto et al., The Botanical Review, April-June, 2001).

Hexane extracts from bark of Juniperus brevifolia have been disclosed to include abietane-type diterpenoids with alcohol function, fatty acids and sterols (Seca et al., Nat Prod Res. 2008; 22(11):975-83).

Resin obtained from Tetraclinis articulata is known as gum sandarac or sandarac gum, and has been disclosed to contain mainly diterpene derivatives, including sandaracopimaric acid, sandaracopimarol, and 4-epidehydroabietic acid (J. Food Hyg. Soc. Jpn. 47(2)). Gum sandarac is also known for use as a food additive.

Specific terpene compounds have been associated with certain therapeutic activities.

Juniperus communis extracts were disclosed to have anti-mycobacterial activity, attributable to the presence of the sesquiterpene longifolene and the diterpenes totarol and trans-communic acid identified therein (Gordien et al., J Ethnopharmacol. 2009 Dec. 10; 126(3):500-5).

WO 2008/061754 discloses certain tricyclic diterpenes, and use thereof as antidepressants, and for the treatment of disorders connected to impaired or reduced neurotransmission, such as disturbed neurotransmission occurring as comorbidity in cardiovascular diseases, strokes, cancer, Alzheimer disease and Parkinson disease.

WO 2008/061720 discloses the use of certain tricyclic diterpenes for the treatment, co-treatment or prevention of inflammatory disorders and joint disorders.

WO 2011/030158 discloses an antimicrobial composition comprising a terpenoid or a derivative thereof, and an antimicrobial agent, wherein the terpenoid may be derived from Cupressaceae, and wherein the terpenoid may be a diterpenoid or a triterpenoid. According to the disclosure, the diterpenoid may be selected from dehydroabietic acid; abietic acid; pimaric acid; kaurenoic acid; ent-3-p-hydroxykaurenoic acid; salvic acid; torarol; 18-acetoxy-cis-cleroda-3,13-Z-dien-15-oic acid; abietinol (7,13-abietadien-18-ol); dehydroabieticylguanidines; pisiferic acid; ferruginol; isopimaric acid; 7-oxo-dehydroabietic acid; 7-hydroxy-dehydroabietic acid; and 13-hydroxy-podocarpa-8,11,13-trien-18-oic acid; and the triterpenoid may be selected from ursolic acid, oleanolic acid, betulinic acid, moronic acid and lupeol.

WO 2010/030054 discloses a composition for preventing or treating hyperlipidemia, fatty liver, diabetes or obesity, wherein the composition comprises as an active ingredient a sesquiterpene inter alia cedrene epoxide, methyl cedryl ether, methyl cedryl ketone or cedrenol, or a pharmaceutically effective amount of an extract of the genus Cupressus.

WO 2002/013840 discloses use of an essential oil, a monoterpene derived thereof, a metabolite and/or a chemically related species in the preparation of a pharmaceutical, cosmetical or nutritional composition for treating a disease or condition involving bone resorption, such as Paget's disease, tumor-induced bone disease or osteoporosis.

WO 2010/100650 and WO 2010/100651 of some of the present inventors disclose isolated fractions of mastic gum and uses thereof in treating impaired neurological function, such as that associated with vascular dementia, senile dementia or Alzheimer's disease. According to these disclosures, the isolated fraction is characterized in that it is soluble in at least one polar organic solvent and in at least one non-polar organic solvent, and comprises polymeric myrcene.

WO 2011/051945 of some of the present inventors disclose use of oligomeric and polymeric forms of the monoterpene compounds alloocimene, limonene, alpha-pinene, beta-pinene, geranyl acetate, alpha-phellandrene, gamma-terpinene, 3-carene and 2-carene for treating degenerative neurological conditions and skin disorders.

There remains an unmet need for botanical products that may be used as active ingredients in pharmaceutical compositions for treating unmet medical needs, such as fibrotic conditions.

### SUMMARY OF THE INVENTION

The present invention provides fractions isolated from trees of the Cupressaceae family, which have surprisingly been found to exhibit the activities of promoting neuronal cell differentiation and inhibiting fibroblast migration. Accordingly, it is disclosed for the first time that Cupressaceae fractions as described herein and pharmaceutical compositions containing them may be used for treating neurological disorders, for example Alzheimer's disease. It is additionally disclosed for the first time herein that the Cupressaceae fractions and compositions of the invention may be used for treating any undesired fibrotic conditions, both those of a pathological nature, for example pulmonary fibrosis, and those involving esthetic disfigurement, for example hypertrophic scarring. Additional therapeutic applications of the invention include prevention or reduction of gliosis following neuronal tissue injury, prevention of surgical adhesions, and promotion of scar-less wound healing following surgical procedures and recovery from other types of wounds, in particular traumatic wounds. Such beneficial applications have not previously been associated with compositions or extracts derived from the Cupressaceae family.

The fractions disclosed herein are prepared by solvent extraction of Cupressaceae plant material, so as to obtain isolated fractions which are soluble in both polar and non-polar solvents. The teachings of the present invention have been exemplified with extracts prepared from resins of Cupressus sempervirens and Tetraclinis articulata using a two-step or three-step extraction procedure, whereby material in the resin that is soluble only in the polar solvent and remains insoluble in the non-polar solvent, is eliminated.

The fractions may also be obtained from Cupressaceae plant material other than resins, such as bark, fruits, leaves, pollen, seeds, twigs, roots and wood. The fractions of the invention are moreover distinguished over prior art Cupressaceae preparations, the latter of which are extracts prepared only with polar solvents, or only with non-polar solvents, or are oils prepared by distillation processes.

Various kinds of plant material have been extracted. Extracts of the Cupressaceae plant material were found to be non toxic and active in applications disclosed herein.

According to a first aspect, the present invention provides an isolated fraction of Cupressaceae plant material, wherein the fraction is characterized in that it is soluble in at least one polar organic solvent and soluble in at least one non-polar organic solvent, and wherein said fraction is substantially devoid of compounds which are soluble in said polar organic solvent but insoluble in said non-polar organic solvent.

In a particular embodiment, the plant material is selected from the group consisting of resin, bark, exudate, fruit, leaves, pollen, seeds, twigs, roots and wood. Each possibility is a separate embodiment of the invention. In a particular embodiment, the plant material is a resin.

In a particular embodiment, the isolated fraction has an HPLC chromatogram substantially as depicted in FIG. 1A or FIG. 1B. Each possibility is a separate embodiment of the invention.

In a particular embodiment, the isolated fraction has an HPLC chromatogram substantially as depicted in FIG. 4.

In a particular embodiment, the isolated fraction comprises at least one compound selected from the group consisting of a monoterpene, a sesquiterpene, a diterpene, a triterpene, a C15-tropolone and combinations thereof. Each possibility is a separate embodiment of the invention. In a particular embodiment, the isolated fraction comprises at least one compound selected from the group consisting of a monoterpene, a sesquiterpene, a diterpene, a C15-tropolone and combinations thereof. Each possibility is a separate embodiment of the invention.

In a particular embodiment, the sesquiterpene is selected from the group consisting of bisabolol, cedrene, farnesol, longifolene, cadinene, germacrene-D, guaiol, β-caryophyllene and combinations thereof. Each possibility is a separate embodiment of the invention.

In a particular embodiment, the diterpene is selected from the group consisting of sempervirol, totarol, ferruginol, manool, torusolol, torusolal, isoagatholal, agathadiol, communic acid and combinations thereof. Each possibility is a separate embodiment of the invention.

In a particular embodiment, the diterpene is selected from the group consisting of sandaracopimaric acid, sandaracopimarol, 4-epidehydroabietic acid and combinations thereof. Each possibility is a separate embodiment of the invention.

In a particular embodiment, the C-15 tropolone is selected from the group consisting of nootkatin, chanootin, β-thujaplicin and combinations thereof. Each possibility is a separate embodiment of the invention.

In a particular embodiment, the isolated fraction comprises at least one compound selected from the group consisting of a sesquiterpenoid, a diterpenoid, a triterpenoid and combinations thereof. Each possibility is a separate embodiment of the invention.

In a particular embodiment, the sesquiterpenoid is selected from the group consisting of famesanes, bisabolanes, eudesmanes, cadalanes, guaianes, ylanganes, eremophilanes, himachalanes, germacranes, bicyclogermacranes, humulanes, derivatives of any of the aforementioned, and combinations thereof. Each possibility is a separate embodiment of the invention.

In a particular embodiment, the diterpenoid is selected from the group consisting of totaranes, phenolic abietanes, abietanes, labdanes, clerodanes, pimaranes, isopimaranes, rimuene, beyeranes, cembranes, kauranes, phyllocladanes, derivatives of any of the aforementioned, and combinations thereof. Each possibility is a separate embodiment of the invention.

In a particular embodiment, the triterpenoid is selected from the group consisting of chameacydines and derivatives thereof.

In a particular embodiment, the isolated fraction comprises at least one compound selected from the group consisting of sempervirol, totarol, ferruginol, manool, torusolol, torusolal, isoagatholal, agathadiol, nootkatin, chanootin and combinations thereof. Each possibility is a separate embodiment of the invention.

In a particular embodiment, the isolated fraction comprises totarol.

In some embodiments, the isolated fraction comprises communic acid. In some embodiments, the isolated fraction consists of communic acid. In some embodiments, the communic acid is a racemic mixture. In some embodiments, the communic acid comprises a mixture of Z and E isomers. In some embodiments, the communic acid comprises or consists of a mixture of E- and Z-communic acid (E:Z≈3:1). In some embodiments, the communic acid comprises or consists of the E-stereoisomer. In some embodiments, the communic acid comprises or consists of the Z-stereoisomer. Each possibility is a separate embodiment.

In a particular embodiment, the isolated fraction is substantially devoid of terpene compounds which are soluble in said polar organic solvent and insoluble in said non-polar organic solvent. In a particular embodiment, the isolated fraction is substantially devoid of α-funebrene.

In a particular embodiment, the isolated fraction comprises totarol, and is substantially devoid of α-funebrene.

In a particular embodiment, the isolated fraction comprises a plurality of terpene compounds, wherein said plurality comprises totarol.

In a particular embodiment, the isolated fraction comprises a plurality of diterpene compounds, wherein said plurality comprises totarol, and further wherein said isolated fraction is substantially devoid of α-funebrene.

In a particular embodiment, the isolated fraction comprises at least one compound selected from the group consisting of sandaracopimaric acid, sandaracopimarol, 4-epidehydroabietic acid and combinations thereof. Each possibility is a separate embodiment of the invention.

In a particular embodiment, the isolated fraction is substantially devoid of terperioid compounds which are soluble in said polar organic solvent and insoluble in said non-polar organic solvent.

In a particular embodiment, the fraction of the invention is for treating impaired neurological function. In a particular embodiment, there is provided a use of the fraction of the invention for preparation of a medicament for treating impaired neurological function. In a particular embodiment, there is provided a method of treating impaired neurological function, the method comprising administering to a subject in need thereof an effective amount of the fraction of the invention, thereby treating impaired neurological function.

In a particular embodiment, the impaired neurological function comprises a decrease in a function selected from the group consisting of cognitive function, sensory function, motor function and combinations thereof.

In particular embodiments, the impaired neurological function is associated with a condition or disease selected from the group consisting of Alzheimer's disease, amyotrophic lateral sclerosis (ALS), multiple sclerosis, Parkinson's disease, vascular dementia and senile dementia. Each possibility represents a separate embodiment of the invention.

In particular embodiments, the impaired neurological function is associated with trauma or stroke. In a particular embodiment, the stroke is associated with at least one of cerebral ischemia, subarachnoid hemorrhage and intracerebral hemorrhage.

In a particular embodiment, the fraction is for treating impaired neurological function associated with Alzheimer's disease.

In a particular embodiment, the fraction is for treating impaired neurological function associated with vascular dementia.

In a particular embodiment, the fraction is for treating impaired neurological function associated with senile dementia.

In a particular embodiment, the fraction is for treating impaired neurological function associated with amyotrophic lateral sclerosis (ALS).

In a particular embodiment, the fraction is for treating impaired neurological function associated with multiple sclerosis.

In a particular embodiment, the fraction is for treating impaired neurological function associated with at least one of cerebral ischemia, subarachnoid hemorrhage and intracerebral hemorrhage.

In a particular embodiment, the impaired neurological function is due to exposure to a drug, such as an anesthetic.

In a particular embodiment, the fraction of the invention is for preventing or treating a fibrotic condition. In a particular embodiment, there is provided a use of the fraction of the invention for preparation of a medicament for preventing or treating a fibrotic condition. In a particular embodiment, there is provided a method of preventing or treating a fibrotic condition, the method comprising administering to a subject in need thereof an effective amount of the fraction of the invention, thereby preventing or treating a fibrotic condition.

In a particular embodiment, the fibrotic condition is selected from the group consisting of arterial fibrosis, arthrofibrosis, bladder fibrosis, breast fibrosis, cardiac fibrosis, endomyocardial fibrosis, liver fibrosis, lymph node fibrosis, mediastinal fibrosis, muscle fibrosis, myelofibrosis, nephrogenic systemic fibrosis, pancreatic fibrosis, pleural fibrosis progressive massive fibrosis, pulmonary fibrosis, renal fibrosis, retroperitoneal fibrosis, skin fibrosis, thyroid fibrosis, cirrhosis, vascular stenosis, restenosis, and chronic obstructive pulmonary disease (COPD). Each possibility represents a separate embodiment of the invention.

In a particular embodiment, the fibrotic condition is selected from the group consisting of scleroderma, a fibromatosis and hypertrophic scarring. Each possibility represents a separate embodiment of the invention. In a particular embodiment, the hypertrophic scarring is selected from the group consisting of post-injury scarring and keloid scar. Each possibility represents a separate embodiment of the invention.

In a particular embodiment, the fraction of the invention is for preventing or reducing scar formation at a wound site. In a particular embodiment, there is provided a use of the fraction of the invention for preparation of a medicament for preventing or reducing scar formation at a wound site. In a particular embodiment, there is provided a method of preventing or reducing scar formation at a wound site, the method comprising administering to a wound site in a subject in need thereof an effective amount of the fraction of the invention, thereby preventing or reducing scar formation at a wound site.

In a particular embodiment, the wound site comprises a wound selected from the group consisting of a burn, an amputation wound, a split-skin donor graft, a skin graft donor site, a medical device implantation site, a bite wound, a frostbite wound, a puncture wound, a shrapnel wound and a surgical wound.

In a particular embodiment, the fraction of the invention is for preventing or treating a surgical adhesion.

In a particular embodiment, the fraction of the invention is for preventing or reducing gliosis.

In a particular embodiment, the gliosis is associated with anoxic injury.

In a particular embodiment, the gliosis is associated with a neurodegenerative disorder selected from the group consisting of Alzheimer's disease, Korsakoffs syndrome, multiple system atrophy, prion disease, multiple sclerosis, AIDS dementia complex, Parkinson's disease, ALS and Huntington's disease. Each possibility represents a separate embodiment of the invention.

In a particular embodiment, the fraction of the invention is for treating tissue damage. In a particular embodiment, the tissue damage is associated with an injury or insult selected from the group consisting of a myocardial infarction, a pulmonary embolism, a cerebral infarction, peripheral artery occlusive disease, a hernia, a splenic infarction, a venous ulcer, an axotomy, a retinal detachment, and a surgical procedure

In particular embodiments of the methods and uses of the invention, the isolated fraction or pharmaceutical composition comprises totarol, and is substantially devoid of and α-funebrene. Each possibility represents a separate embodiment of the invention.

In a particular embodiment, the isolated fraction is obtained by a process comprising the steps of:
(a) treating a Cupressaceae resin with a polar organic solvent;
(b) isolating a fraction soluble in said polar organic solvent;
(c) optionally removing said polar organic solvent;
(d) treating the soluble fraction obtained in step (b) or (c) with a non-polar organic solvent,
(e) isolating a fraction soluble in said non-polar organic solvent; and
(f) optionally removing said non-polar organic solvent;
wherein steps (d) to (f) may precede steps (a) to (c).

In particular embodiments, steps (a) to (c) are carried out prior to steps (d) to (f); or steps (d) to (f) are carried out prior to steps (a) to (c). In particular embodiments, (a) to (c) and/or steps (d) to (f) are repeated for a multiplicity of cycles.

In a particular embodiment, either or both of steps (c) and (f) comprise removing the solvent by a means selected from the group consisting of rotary evaporation, application of high vacuum and a combination thereof. In a particular embodiment, the process further comprises the step of size fractionating the fraction obtained by said process.

Polar organic solvents suitable for use in the invention may be selected from an alcohol, an ether, an ester, an amide, an aldehyde, a ketone, a nitrile, and combinations thereof.

Specific examples of suitable polar organic solvents include methanol, ethanol, propanol, isopropanol, 1-butanol, 2-butanol, sec-butanol, t-butanol, 1-pentanol, 2-pentanol, 3-pentanol, neopentanol, 3-methyl-1-butanol, 2-methyl-1-butanol, 3-methyl-2-butanol, 2-methyl-2-butanol, ethyleneglycol, ethyleneglycol monomethyl ether, diethyl ether, methylethyl ether, ethylpropyl ether, methylpropyl ether, 1,2-dimethoxyethane, tetrahydrofuran, dihydrofuran, furan, pyran, dihydropyran, tetrahydropyran, methyl acetate, ethyl acetate, propyl acetate, acetaldehyde, methylformate, ethylformate, ethyl propionate, methyl propionate, dichloromethane, chloroform, dimethylformamide, acetamide, dimethylacetamide, N-methylpyrrolidone, acetone, ethylmethyl ketone, diethyl ketone, acetonitrile, propionitrile, and combinations thereof. In particular embodiments, the polar organic solvent is ethanol.

Non-polar organic solvents suitable for use in the invention may be selected from acyclic or cyclic, saturated or unsaturated aliphatic hydrocarbons and aromatic hydrocarbons, each of which is optionally substituted by one or more halogens, dialkyl ethers, alkyl-aryl ethers, diaryl ethers and combinations thereof. In particular embodiments, the non-polar organic solvent is selected from C5-C10 alkanes, chlorinated hydrocarbons, C5-C10 cycloalkanes, C6-C14 aromatic hydrocarbons, dialkyl ethers and C7-C14 perfluoroalkanes, and combinations thereof.

In particular embodiments, the non-polar organic solvent is selected from pentanes, hexanes, heptanes, octanes, nonanes, decanes, cyclopentane, cyclohexane, cycloheptane, benzene, toluene, xylene, dichloromethane, chloroform and isomers and mixtures thereof.

In particular embodiments, the C5-C10 alkane is selected from the group consisting of pentane, hexane, heptane, octane, nonane, decane, cyclohexane, and isomers and mixtures thereof.

In particular embodiments, the non-polar organic solvent is hexane.

In a particular embodiment, the polar organic solvent comprises ethanol and the non-polar organic solvent comprises hexane.

In a particular embodiment, the invention provides a pharmaceutical composition comprising an effective amount of an isolated fraction of Cupressaceae plant material according to the invention, and a pharmaceutically acceptable carrier. In a particular embodiment, the pharmaceutical composition consists of an effective amount of the isolated fraction, and a pharmaceutically acceptable carrier. In a particular embodiment, the isolated fraction is prepared from a Cupressaceae resin.

In a particular embodiment, the pharmaceutical composition consists of an effective amount of an isolated fraction of a Cupressaceae resin, and a pharmaceutically acceptable carrier. In a particular embodiment, the pharmaceutical composition comprises an effective amount of an isolated fraction of a Cupressaceae resin, and a pharmaceutically acceptable carrier. In a particular embodiment, the pharmaceutical composition is substantially devoid of plant-derived material other than the isolated fraction of Cupressaceae plant material according to the invention. In a particular embodiment, an isolated fraction of a Cupressaceae resin is the sole active ingredient in the pharmaceutical composition.

In a particular embodiment, the composition comprises from about 0.01 to about 25% (w/w) of the isolated fraction of a Cupressaceae resin, based on the total weight of the composition. In a particular embodiment, the composition comprises from about 0.01 to about 12% (w/w) of the isolated fraction of a Cupressaceae resin, based on the total weight of the composition.

In a particular embodiment, the composition comprises at least one compound selected from the group consisting of sempervirol, totarol, ferruginol, manool, torusolol, torusolal, isoagatholal, agathadiol, nootkatin, chanootin, sandaracopimaric acid, sandaracopimarol, 4-epidehydroabietic acid, pharmaceutically acceptable salts of any of the aforementioned acids, and combinations thereof. Each possibility is a separate embodiment of the invention.

In a particular embodiment, the Cupressaceae plant material is from a tree or plant classified in a subfamily selected from the group consisting of Cupressoideae, Callitroideae, Taiwanioideae, Taxodioideae, Athrotaxidioideae, Sequoioideae and Cunninghamhioideae. Each possibility is a separate embodiment of the invention.

In a particular embodiment, the Cupressaceae plant material is from a tree or plant classified in a genus selected from the group consisting of Tetraclinis, Cupressus, Juniperus, Actinostrobus, Athrotaxis, Austrocedrus, Callitris, Callitropsis, Calocedrus, Chamaecyparis, Cryptomeria, Cunninghamia, Diselma, Fitzroya, Fokienia, Glyptostrobus, Libocedrus, Neocallitropsis, Papuacedrus, Pilgerodendron, Platycladus, Sequoia, Metasequoia, Sequoiadendron, Taxodium, Taiwania, Thuja, Thujopsis and Widdringtonia. Each possibility is a separate embodiment of the invention.

In a particular embodiment, the Cupressaceae plant material is from a species selected from the group consisting of Tetraclinis articulata, Cupressus sempervirens, Cupressus funebris, Cupressus atlantica, Cupressus cashmeriana, Cupressus duclouxiana, Cupressus torulosa, Cupressus gigantea, Cupressus dupreziana, Cupressus chengiana, Cupressus nootkatensis, Cupressus sargentii, Cupressus pigmaea, Cupressus macnabiana, Cupressus lusitanica, Cupressus arizonica, Cupressus bakeri, Cupressus goveniana, Cupressus forbesii, Cupressus guadalupensis, Cupressus marcrocarpa, Cupressus abramsiana, Cupressus stephensonii, Juniperus communis, Juniperus conferta, Juniperus rigida, Juniperus brevifolia, Juniperus phoenicea, Juniperus cedrus, Juniperus deltoides, Juniperus formosana, Juniperus lutchuensis, Juniperus navicularis, Juniperus oxycedrus, Juniperus macrocarpa, Juniperus chinensis, Juniperus convallium, Juniperus excelsa, Juniperus polycarpos, Juniperus foetidissima, Juniperus indica, Juniperus komarovii, Juniperus phoenicea, Juniperus procera, Juniperus procumbens, Juniperus pseudosabina, Juniperus recurva, Juniperus sabina, Juniperus saltuaria, Juniperus semiglobosa, Juniperus squamata, Juniperus thuriftra, Juniperus tibetica and Juniperus wallichiana. Each possibility is a separate embodiment of the invention.

In a particular embodiment, the Cupressaceae plant material is from a species is selected from the group consisting of Tetraclinis articulata, Cupressus sempervirens and Juniperus communis.

In a particular embodiment, the Cupressaceae plant material is from Tetraclinis articulata.

In a particular embodiment, the Cupressaceae plant material is from Cupressus sempervirens.

In particular embodiments, the composition is substantially devoid of terpene compounds which are soluble in the polar organic solvent and insoluble in the non-polar organic solvent used to prepare the isolated fraction.

In particular embodiments, the composition is substantially devoid of terpenoid compounds which are soluble in the polar organic solvent and insoluble in the non-polar organic solvent used to prepare the isolated fraction.

In a particular embodiment, the pharmaceutically acceptable carrier comprises at least one oil. In a particular embodiment, the oil is selected from the group consisting of a mineral oil, a vegetable oil and combinations thereof. In a particular embodiment, the vegetable oil is selected from the group consisting of almond oil, canola oil, coconut oil, corn oil, cottonseed oil, grape seed oil, olive oil peanut oil, saffron oil, sesame oil, soybean oil, and combinations thereof. Each possibility represents a separate embodiment of the invention.

In a particular embodiment, the mineral oil is light mineral oil. In a particular embodiment, the vegetable oil is cottonseed oil. In a particular embodiment, the carrier comprises at least one wax. In a particular embodiment, the carrier comprises a combination of at least one oil and at least one wax.

In various embodiments, a composition according to the invention is in a form suitable for administration by a route selected from the group consisting of oral, topical, parenteral and transdermal. Each possibility represents a separate embodiment of the invention.

In particular embodiments, the composition is in a form suitable for administration by injection. In various embodiments, the composition is a parenteral formulation for administration by a route selected from the group consisting of intravenous, intramuscular, subcutaneous, intradermal, intraperitoneal, intraarterial, intracerebral, intracerebroventricular, intraosseus and intrathecal. Each possibility represents a separate embodiment of the invention.

In various embodiments, the composition is a topical formulation for administration by a route selected from the group consisting of dermal, vaginal, rectal, inhalation, intranasal, ocular, auricular and buccal. Each possibility represents a separate embodiment of the invention.

In particular embodiments, the composition is in a form suitable for cosmetic or dermatologic administration.

In particular embodiments, the pharmaceutical composition is in a form selected from the group consisting of a capsule, a tablet, a liposome, a suppository, a suspension, an ointment, a cream, a lotion, a solution, an emulsion, a film, a cement, a powder, a glue, an aerosol and a spray. Each possibility represents a separate embodiment of the invention.

In a particular embodiment, the composition is disposed on the article of manufacture in the form of a coating. In a particular embodiment, the article of manufacture comprises a vessel, wherein the composition is disposed within the vessel.

In a particular embodiment, the composition is suitable for administration by a means selected from the group consisting of electroporation, sonication, radio frequency, pressurized spray and combinations thereof.

In a particular embodiment, the composition of the invention is for inducing or promoting tissue repair following an injury or insult.

Other objects, features and advantages of the present invention will become clear from the following description and drawings.

### BRIEF DESCRIPTION OF THE DRAWINGS

FIGS. 1A-J show HPLC characterization of fractions obtained from Cupressus sempervirens. FIGS. 1A and 1B are HPLC chromatograms of fractions (RPh-CY-DS) obtained by extraction of two separate batches of resin obtained from different Cupressus sempervirens trees growing in the Carmel region in northern Israel. The HPLC-method used to obtain the chromatograms is depicted in FIG. 1C. FIG. 1D shows a chromatogram of the totarol reference compound. FIGS. 1E-G show chromatograms obtained in a spiking experiment with totarol. FIG. 1E shows analysis of a diluted sample (RPh-CY), and FIG. 1F shows analysis of RPh-CY spiked with a commercial preparation of totarol. FIG. 1G shows an overlay of FIGS. 1E and 1F. FIGS. 1H-1K show HPLC analyses confirming the absence of α-funebrene in RPh-CY-DS. FIG. 1H shows a chromatogram of an RPh-CY-DS sample before spiking with an α-funebrene reference compound. FIG. 1I shows a chromatogram of the α-funebrene reference compound. FIG. 1J shows analysis of RPh-CY-DS spiked with the α-funebrene reference compound. FIG. 1K shows a magnified view of an overlay of FIGS. 1H-J in the region 60.5-65 min retention time, confirming that no α-funebrene is present in RPh-CY-DS.

FIGS. 2A-C show the effects of treatment with RPh-CY on ARPE-19 cells. FIG. 2A shows cells following treatment (48 hr) with RPh-CY (10% w/w) prepared from the fraction shown in FIG. 1A. FIG. 2B shows cells following treatment with RPh-CY prepared from the fraction shown in FIG. 1B formulated to a concentration of 10% (w/w) in cottonseed oil. FIG. 2C shows cells that were treated with cottonseed oil alone (vehicle control).

FIGS. 3A-B show the effects of treatment with CY-POLAR on ARPE-19 cells. FIG. 3A shows ARPE-19 cells after treatment (48 hrs) with CY-POLAR (2.5% in isopropanol). FIG. 3B shows control ARPE-19 cells treated with isopropanol only.

FIG. 4 shows an HPLC chromatogram of an isolated fraction (RPh-SA-DS), obtained from the resin of Tetraclinis articulata (gum sandarac) as described in Example 3.

FIGS. 5A-B show the effects of treatment with RPh-SA on ARPE-19 cells. FIG. 5A shows cells treated with RPh-SA, prepared according to Example 6. FIG. 5B shows cells treated with cottonseed oil alone (vehicle).

FIG. 6A-B show the effects of treatment with SA-POLAR on ARPE-19 cells. FIG. 6A shows cells after treatment (48 hrs) with SA-POLAR (2.5% in isopropanol). FIG. 6B shows control ARPE-19 cells treated only with isopropanol.

FIG. 7A-B show the ¹H-NMR spectrum (FIG. 7A) and ¹³C-NMR spectrum (FIG. 7B) of the mixture of the ≈3:1 E- and Z-communic acid, respectively, isolated from RPh-SA-DS.

FIG. 8 shows the effects of treatment with RPh-CMA on ARPE-19 cells following treatment (24 hr) with RPh-CMA (10% w/w) prepared from the fraction shown in FIG. 4. (See Examples 4 and 5.)

FIG. 9 shows the results of cytotoxicity studies with RPh-CY and RPh-SA, based on the data in Tables 2 and 3.

FIG. 10 shows the results of the scratch assay, expressed as the decrease of the scratch gap-width as a function of time.

FIGS. 11A-E show representative photographs taken at different time points (OT, 24 hr, 48 hr and 55 hr) in the scratch assay, showing cells treated with RPh-CY (FIG. 11A); RPh-SA (FIG. 11B); oil vehicle only (FIG. 11C); 1% FCS (FIG. 11D); and untreated cells (FIG. 11E).

### DETAILED DESCRIPTION OF THE INVENTION

The inventors of the present invention have surprisingly found that specific extract fractions isolated from Cupressaceae plant material, in particular resins, have activity in ameliorating impaired neurological function, and in inhibiting fibroblast migration.

Accordingly, isolated fractions of Cupressaceae resins as described herein may be employed as an active ingredient in a pharmaceutical composition for a number of therapeutic indications.

Advantageously, the compositions of the invention may be used in methods of treating impaired neurological function. Upon contact with cells of both human and non-human subjects, the composition induces cell differentiation in a wide array of tissues, cell compartments and cell lineages, including skin, endothelium, mucous membranes, bones, tendons and cartilage. In addition, the cell differentiation activity of the pharmaceutical composition may be exploited for promoting in vivo incorporation of medical devices, implants and organ transplants.

Without wishing to be bound by any particular theory or mechanism of action, the activity of isolated Cupressaceae extracts for induction of neuronal cell differentiation, as disclosed herein, renders the present invention useful for reformation of inter-neuronal junctions and overcoming defective inter-neuronal communication in brain and neural tissue affected by pathologies associated with inadequate synaptic formation. This pathology underlies many nervous system pathologies, including for example Alzheimer's disease. The invention is further useful for promoting rejuvenation and regeneration of a large number of cells and tissues, including those of ectodermal, mesodermal and endodermal origin.

Moreover, it is disclosed for the first time herein, that Cupressaceae fractions and pharmaceutical compositions containing them may be used for treating fibrotic conditions, including fibrosis in various organs, for example pulmonary fibrosis. Similarly, the subject fractions and compositions may be used to prevent or reduce scar formation at wound sites, including surgical wounds and traumatic wounds. In addition, the fractions and compositions disclosed herein may be effectively used for preventing or reducing gliosis following injury of neuronal tissues.

### DEFINITIONS

As used herein, the term “Cupressaceae” is used to refer to any tree or plant classified in the family Cupressaceae, including all subfamilies, genera, species, subspecies and cultivars thereof. Subfamilies of Cupressaceae include, but are not limited to Cupressoideae, Callitroideae, Taiwanioideae, Taxodioideae, Athrotaxidioideae, Sequoioideae or Cunninghamhoideae. Genera of Cupressaceae include, but are not limited to Actinostrobus, Athrotaxis, Austrocedrus, Callitris, Callitropsis, Calocedrus, Chamaecyparis, Cryptomeria, Cunninghamia, Diselma, Fitzroya, Fokienia, Glyptostrobus, Libocedrus, Neocallitropsis, Papuacedrus, Pilgerodendron, Platycladus, Sequoia, Metasequoia, Sequoiadendron, Taxodium, Taiwania, Tetraclinis, Thuja, Thujopsis and Widdringtonia.

Exemplary species of Cupressaceae include, but are not limited to Tetraclinis articulata, Cupressus sempervirens, Cupressus funebris, Cupressus atlantica, Cupressus cashmeriana, Cupressus duclouxiana, Cupressus torulosa, Cupressus gigantea, Cupressus dupreziana, Cupressus chengiana, Cupressus nootkatensis, Cupressus sargentii, Cupressus pigmaea, Cupressus macnabiana, Cupressus lusitanica, Cupressus arizonica, Cupressus bakeri, Cupressus goveniana, Cupressus forbesii, Cupressus guadalupensis, Cupressus marcrocarpa, Cupressus abramsiana, Cupressus stephensonii, Juniperus communis, Juniperus conferta, Juniperus rigida, Juniperus brevifolia, Juniperus phoenicea, Juniperus cedrus, Juniperus deltoides, Juniperus formosana, Juniperus lutchuensis, Juniperus navicularis, Juniperus oxycedrus, Juniperus macrocarpa, Juniperus chinensis, Juniperus convallium, Juniperus excelsa, Juniperus polycarpos, Juniperus foetidissima, Juniperus indica, Juniperus komarovii, Juniperus phoenicea, Juniperus procera, Juniperus procumbens, Juniperus pseudosabina, Juniperus recurva, Juniperus sabina, Juniperus saltuaria, Juniperus semiglobosa, Juniperus squamata, Juniperus thurifera, Juniperus tibetica and Juniperus wallichiana.

As used herein, the term “Cupressaceae plant material” refers to any part of or exudate from a plant classified in the family Cupressaceae, including resin, bark, fruits, leaves, pollen, seeds, twigs, roots and wood.

As used herein, the term “an isolated fraction of Cupressaceae plant material” refers to a fraction obtained following extraction of Cupressaceae plant material using at least one polar and at least one non-polar organic solvent, or combinations thereof. The isolated fraction of the invention is soluble in both of said polar and non-polar organic solvents used for its preparation.

As used herein, the term “Cupressaceae resin” refers to a tree resin (also known as an oleoresin) obtained as an exudate from any tree or plant classified in the family Cupressaceae, including for example the resin known as gum sandarac or sandarac gum which is obtained from Tetraclinis articulata.

As used herein, the term “degree of purity” refers to the content of a specified chemical compound in a preparation, expressed as a percentage on a weight per weight basis of the specified chemical compound relative to other chemical compounds in the preparation.

As used herein, “terpene compounds” refers to isoprene-containing hydrocarbons, having isoprene units (CH₂═C(CH₃)—CH═CH₂) in a head-to-tail orientation. Terpene hydrocarbons in general, have the molecular formula (C₅H₈)ₙ, and include hemiterpenes, (C5), monoterpenes (C10), sesquiterpenes (C15), diterpenes (C20), triterpenes (C30), and tetraterpenes (C40) which respectively have 1, 2, 3, 4, 6 and 8 isoprene units. Terpenes may be further classified as acyclic or cyclic. Examples of monoterpenes include α-pinene and β-pinene, sabinene and limonene. Examples of sesquiterpenes include bisabolol, farnesol, longifolene, cadinene and cedrene. Examples of diterpenes include totarol, ferruginol, agathadiol and (+)-sempervirol.

As used herein, “terpenoids” and “terpenoid compounds” interchangeably refer to terpene-related compounds which contain oxygen in addition to isoprene units, and thus include alcohols, aldehydes and ketones. Terpenoids are subdivided according to the number of carbon atoms in a manner similar to terpenes, and thus include hemiterpenoids, (C5), monoterpenoids (C10), sesquiterpenoids (C15), diterpenoids (C20), triterpenoids (C30), and tetraterpenoids (C40) which respectively have 1, 2, 3, 4, 6 and 8 isoprene units. The skeleton of terpenoids may differ from strict additivity of isoprene units by the loss or shift of a fragment, generally a methyl group. Examples of monoterpenoids include camphor, eugenol and borneol. Examples of diterpenoids include phytol and taxol. Examples of triterpenoids include squalene and lanosterol.

As used herein, “terpenoic acids” refer to terpenoid compounds containing at least one carboxylic acid group. The terpenoic acids may additionally contain one or more other oxygen-containing functional groups comprising hydroxyl, keto, aldehyde, ether (cyclic and non-cyclic) and ester (cyclic and non-cyclic) groups.

As used herein, “sesquiterpenoic acids” refer to sesquiterpenoid compounds containing at least one carboxylic acid group. The sesquiterpenoic acids may additionally contain one or more other oxygen-containing functional groups comprising hydroxyl, keto, aldehyde, ether (cyclic and non-cyclic) and ester (cyclic and non-cyclic) groups.

As used herein, “diterpenoic acids” refer to diterpenoid compounds containing at least one carboxylic acid group. The diterpenoic acids may additionally contain one or more other oxygen-containing functional groups comprising hydroxyl, keto, aldehyde, ether (cyclic and non-cyclic) and ester (cyclic and non-cyclic) groups.

As used herein, “triterpenoic acids” refer to triterpenoid compounds containing at least one carboxylic acid group. The triterpenoic acids may additionally contain one or more other oxygen-containing functional groups comprising hydroxyl, keto, aldehyde, ether (cyclic and non-cyclic) and ester (cyclic and non-cyclic) groups.

As used herein, “an oligomeric form of a terpenoic acid” refers to an oligomeric terpenoid acid in which the monomeric units are either of the same terpenoic acid or of different terpenoic acids, and are joined in any possible arrangements, and are connected one to another through any possible bond or functional group, such as a C—C bond, an ester group or an ether group.

As used herein, “substantially devoid” means that a preparation or pharmaceutical composition according to the invention that generally contains less than 3% of the stated substance, preferable less than 1% and most preferably less than 0.5%. As used herein, “therapeutically effective amount” refers to that amount of a pharmaceutical ingredient which substantially induces, promotes or results in a desired therapeutic effect.

As used herein, “pharmaceutically acceptable carrier” refers to a diluent or vehicle which is used to enhance the delivery and/or pharmacokinetic properties of a pharmaceutical ingredient with which it is formulated, but has no therapeutic effect of its own, nor does it induce or cause any undesirable or untoward effect or adverse reaction in the subject.

As used herein, “cell differentiation” refers to the process in which a less specialized cell becomes a more specialized cell. Cell differentiation may be established on the basis of changes in any of a number of cellular characteristics, including but not limited to size, shape, organelle appearance, membrane potential, metabolic activity, and responsiveness to signals. A particular “grade” may be given to a cell type to describe the extent of differentiation.

As used herein, “impaired neurological function” refers to a decline or decrease in at least one of sensory, cognitive or motor function, as compared to a previous level of function or activity, and/or as compared to non-impaired individuals matched according to accepted criteria.

As used herein, “life span extension” refers to prolongation of life span of an animal beyond the generally expected life span of said animal in the absence of treatment with the compositions and methods of the invention.

As used herein, the terms “fibrotic condition” refers to a benign or pathological condition characterized by excess fibrous connective tissue in an organ or tissue, present in an amount and/or form that is substantially different from that normally found in such organ or tissue, and that is formed in a reparative or reactive process.

As used herein, “gliosis” refers to proliferation of astrocytes in damaged areas of the central nervous system (CNS). In particular embodiments, gliosis comprises formation of a glial scar.

Numerical values stated herein are to be understood as the stated value +/−10%.

### Isolated Fractions of Cupressaceae Plant Material

The present invention provides isolated fractions of Cupressaceae plant material. Suitable Cupressaceae trees and plants include those classified in a Cupressaceae subfamily such as Cupressoideae, Callitroideae, Taiwanioideae, Taxodioideae, Athrotaxidioideae, Sequoioideae and Cunninghamhoideae.

It is to be understood that the fractions of the invention generally comprise mixtures of specific compounds, the relative proportions of which may vary, depending on the Cupressaceae genus or species from which the fraction is obtained and possibly other factors, such as environmental and climatic variations during growth of the source tree.

Analytical methods for determining the chemical composition of the isolated fractions obtained from Cupressaceae resins and other materials or parts, include nuclear magnetic resonance (for example ¹H-NMR and ¹³C-NMR), viscometry, various mass spectrometry methods (for example MALDI-TOF), combination methods such as Liquid Chromatography-Mass spectrometry (LC-MS), UV-VIS spectrophotometry, IR and FT-IR spectrophotometry and other methods as are known in the art.

Several methods may be used for obtaining isolated fractions of Cupressaceae plant material. By way of a general description, resin or other material from a Cupressaceae species, for example Tetraclinis articulata or Cupressus sempervirens is combined in a suitable vessel with a suitable solvent, usually a polar solvent. Suitable polar solvents include ethanol, methanol, propanol, isopropanol, 1-butanol, 2-butanol, sec-butanol, t-butanol, 1-pentanol, 2-pentanol, 3-pentanol, neopentanol, 3-methyl-1-butanol, 2-methyl-1-butanol, 3-methyl-2-butanol, 2-methyl-2-butanol, ethyleneglycol, ethyleneglycol monomethyl ether, diethyl ether, methylethyl ether, ethylpropyl ether, methylpropyl ether, 1,2-dimethoxyethane, tetrahydrofuran, dihydrofuran, furan, pyran, dihydropyran, tetrahydropyran, methyl acetate, ethyl acetate, propyl acetate, acetaldehyde, methylformate, ethylformate, ethyl propionate, methyl propionate, dichloromethane, chloroform, dimethylformamide, acetamide, dimethylacetamide, N-methylpyrrolidone, acetone, ethylmethyl ketone, diethyl ketone, acetonitrile, propionitrile, and combinations thereof.

The Cupressaceae resin and the polar solvent are preferably combined such that the polar solvent is in large excess, for example 10:1 or 20:1. The mixture may be periodically or continuously agitated over a period ranging from a few minutes to a number of hours. The mixture may be allowed to stand for a period ranging from a few minutes to two weeks in order to facilitate the precipitation of insoluble material. The solvent may be decanted without any treatment, or optionally the mixture may be first subjected to low speed centrifugation, for example at 100 to 2000 rpm, as is known in the art. Alternatively, the insoluble material may be removed by suction filtration, for example using a sintered glass filter. The insoluble material is recovered from the extract and a fresh aliquot of solvent is optionally added to the insoluble material, such that the extraction and dissolution process is repeated for a number of cycles, in order to obtain as much as possible of the polar solvent soluble compounds. After the final dissolution step, the extracts containing polar solvent soluble material are combined and the polar solvent is evaporated (for example by using a rotary evaporation as is known in the art), so as to yield polar solvent soluble material, which may be referred to as a crude, or “first step” extract.

The first step extract material is combined with a non-polar organic solvent and extracted by shaking over a period of 2 hours. Suitable non-polar solvents include acyclic or cyclic, saturated or unsaturated aliphatic hydrocarbons and aromatic hydrocarbons, for example, C5-C10 alkanes, C5-C10 cycloalkanes, C6-C14 aromatic hydrocarbons, and combinations thereof. Each of the foregoing may be optionally substituted by one or more halogens, for example, C7-C14 perfluoroalkanes. Particular examples of non-polar organic solvents are pentanes, hexanes, heptanes, octanes, nonanes, decanes, cyclopentane, cyclohexane, cycloheptane, benzene, toluene, xylenes, and isomers and mixtures thereof. Material remaining insoluble or precipitating in the presence of the non-polar solvent is removed and discarded. The non-polar solvent-soluble fraction is then obtained by evaporating the non-polar solvent (for example by rotary evaporation). This fraction may be referred to as purified or “two step” extract, corresponding to an isolated Cupressaceae fraction characterized by the fact that it is soluble in both a polar solvent and a non-polar solvent, while materials which are soluble in the polar solvent but insoluble in the non-polar solvent, have been removed.

The obtained two step extract may be dried further, for example by high vacuum treatment (for example <0.01 mbar for up to several days) to remove residual solvent and other volatile material, weighed and combined with a suitable non-polar organic solvent or other carrier to effect its dissolution. The isolation of such final fractions is disclosed herein in Examples 1 and 3. The obtained fractions may be used directly, or further purified, characterized and/or fractionated using means known in the art, as enumerated above.

In a particular embodiment, the isolated fraction is obtained by a process comprising the steps of:
(a) treating a Cupressaceae resin with a polar organic solvent;
(b) isolating a fraction soluble in said polar organic solvent;
(c) optionally removing said polar organic solvent;
(d) treating the soluble fraction obtained in step (b) or (c) with a non-polar organic solvent,
(e) isolating a fraction soluble in said non-polar organic solvent; and
(f) optionally removing said non-polar organic solvent;
wherein steps (d) to (f) may precede steps (a) to (c).

In particular embodiments, steps (a) to (c) are carried out prior to steps (d) to (f); or steps (d) to (f) are carried out prior to steps (a) to (c). In particular embodiments, (a) to (c) and/or steps (d) to (f) are repeated for a multiplicity of cycles.

In a particular embodiment, either or both of steps (c) and (f) comprise removing the solvent by a means selected from the group consisting of rotary evaporation, application of high vacuum and a combination thereof. In a particular embodiment, the process further comprises the step of size fractionating the fraction obtained by said process.

In a particular embodiment, the process further comprises the steps of:
(g) dissolving the isolated fraction from step (e) or (f) in a suitable organic solvent;
(h) extracting the organic fraction obtained in (g) with a basic aqueous solution so as to obtain a basic aqueous fraction and a second organic fraction;
(i) acidifying the basic aqueous fraction obtained in (h) with an acidic solution so as to obtain an acidic fraction;
(j) extracting the acidic fraction obtained in (i) with a suitable organic solvent so as to obtain an organic acidic fraction;
(k) evaporating the organic solvent from the organic acidic fraction obtained in step (j) so as to obtain an isolated acidic fraction of a Cupressaceae resin.

In a particular embodiment, the process further comprises combining the second organic fraction obtained from step (h) with a fraction obtained in any of steps (i), (j) or (k).

In a particular embodiment, the second organic fraction obtained from step (h) is combined with a fraction obtained in any of steps (i), (j) or (k) in an amount in the range from 0.1 to 100% of the organic fraction obtained from step (h). In particular embodiments, the amount is in the range from 0.5 to 80%; or 1 to 50%; or 2 to 25%; or 0.1 to 10%.

In a particular embodiment, the process described above comprises combining the second organic fraction obtained from step (h) with an isolated fraction obtained in step (j) or step (k). In a particular embodiment, the amount of the second organic fraction obtained from step (h) present that is combined with the isolated fraction obtained in step (j) or step (k) is in the range from about 0.1-20% w/w based on the total weight of the isolated fraction obtained in step (j) or step (k).

In particular embodiments, the organic solvent in step (g) and in step (j) is the same or different, and is independently selected from the group consisting of dialkyl ethers, alkyl-aryl ethers, diaryl ethers, esters, ketones, halogenated hydrocarbons, C5-C14 aromatic hydrocarbons, C5-C14 perfluoroalkanes.

In particular embodiments, the organic solvent comprises a dialkyl ether. In particular embodiments, the organic solvent is diethyl ether.

In a particular embodiment, the basic aqueous solution in step (h) is prepared by dissolving an inorganic base in water.

Suitable inorganic bases include for example, sodium carbonate, potassium carbonate, sodium hydroxide, potassium hydroxide, ammonium hydroxide, sodium bicarbonate, sodium phosphate, lithium hydroxide, lithium carbonate, and potassium phosphate.

The basic aqueous solution used in step (h) is typically made by dissolving a water soluble organic base in water. The organic base may be for example, a water soluble aliphatic or an aromatic amine.

In a particular embodiment, the acidic solution in step (i) comprises an acidic aqueous solution or an acidic non-aqueous solution.

An acidic aqueous solution may be prepared by dissolving an inorganic acid in water; or by dissolving an organic acid in water; or by diluting a concentrated mineral acid solution.

In a particular embodiment, the acidic aqueous solution is a solution of hydrochloric acid or phosphoric acid.

An acidic non-aqueous solution may be prepared by dissolving an organic acid in a non-aqueous organic solvent, for example an alcohol, an ester, an ether, an amide or mixtures thereof. In a particular embodiment, the non-aqueous solvent is methanol, ethanol or a mixture thereof.

In a particular embodiment, the organic acid is selected from the group consisting of formic acid, acetic acid, propionic acid, citric acid, tartaric acid, methane sulphonic acid, and para-toluenesulphonic acid.

Compounds which may be contained in the isolated fraction of the invention include one or more of monoterpenes, sesquiterpenes, diterpenes, triterpenes, C15-tropolones, sesquiterpennoids, diterpenoids, triterpenoids, derivatives of any of the aforementioned and combinations thereof.

Examples of sesquiterpenoid compounds that may be present in the isolated fractions include, but are not limited to famesanes, bisabolanes, eudesmanes, cadalanes, guaianes, ylanganes, eremophilanes, himachalanes, germacranes, bicyclogermacranes, humulanes and derivatives thereof.

Examples of diterpenoid compounds that may be present in the isolated fractions include, but are not limited to totaranes, phenolic abietanes, abietanes, labdanes, clerodanes, pimaranes, isopimaranes, rimuene, beyeranes, cembranes, kauranes, phyllocladanes, and derivatives thereof.

Examples of triterpenoid compounds that may be present in the isolated fractions include, but are not limited to chameacydines and derivatives thereof.

Examples of sesquiterpene compounds that may be present in the isolated fractions include, but are not limited to bisabolol, cedrene, farnesol, longifolene, cadinene, germacrene-D, guaiol and β-caryophyllene.

Examples of diterpene compounds that may be present in the isolated fractions include, but are not limited to sempervirol, totarol, ferruginol, manool, torusolol, torusolal, isoagatholal, and agathadiol. Yet other diterpene compounds that may be present in the isolated fractions include, but are not limited to sandaracopimaric acid, sandaracopimarol, and 4-epidehydroabietic acid, and salts thereof.

Examples of C-15 tropolones present in the isolated fractions include, but are not limited to nootkatin, chanootin and β-thujaplicin.

The invention further encompasses fractions and compositions comprising at least one acid selected from the group consisting of terpenoic acids, sequiterpenoic acids, diterpenoic acids, triterpenoic acids, pharmaceutically acceptable salts of any of the aforementioned acids and combinations thereof.

In some embodiments, the isolated fraction comprises communic acid. In some embodiments, the isolated fraction consists of communic acid. In some embodiments, the communic acid is a racemic mixture. In some embodiments, the communic acid comprises a mixture of Z and E isomers. In some embodiments, the communic acid comprises or consists of a mixture of E- and Z-communic acid (E:Z about 3:1). In some embodiments, the communic acid comprises or consists of the E stereoisomer. In some embodiments, the communic acid comprises or consists of the Z stereoisomer. Each possibility is a separate embodiment.

Preparation of Cuppressaceae extracts is described herein in Examples 1, 3 and 4. Example 1 demonstrates that a fraction isolated from the resin of C. sempervirens by a two step extraction process comprises a combination of terpene compounds including totarol, and does not include α-funebrene. Example 4 demonstrates that a fraction isolated from the resin of Tetraclinis articulate, obtained by a two step extraction process according to Example 3, comprises a mixture of E- and Z-communic acid with an E:Z ratio of approximately about 3:1.

### Pharmaceutical Compositions

The composition for use in the invention comprises a therapeutically effective amount of an isolated fraction of Cupressaceae plant material, and a pharmaceutically acceptable carrier.

Preparation of formulations containing Cupressaceae fractions are described herein in Examples 2, 5 and 6.

A suitable carrier comprises at least one oil, such as for example a mineral oil, a vegetable oil or combinations thereof, optionally in combination with waxes.

The term “mineral oil” refers to a clear colorless nearly odorless and tasteless liquid obtained from the distillation of petroleum. It may also be referred to as white oil, white mineral oil, liquid petrolatum, liquid paraffin or white paraffin oil. In accordance with a particular embodiment of the invention, the mineral oil is light mineral oil, a commercially available product which may be obtained either as a NF (National Formulary) grade product or as a USP (US Pharmacopoeia) grade product. For use in the invention, the mineral oil is preferably free of aromatics and unsaturated compounds.

Suitable vegetable oils include, but are not limited to almond oil, canola oil, coconut oil, corn oil, cottonseed oil, grape seed oil, olive oil peanut oil, saffron oil, sesame oil, soybean oil, or combinations thereof. In a particular embodiment, the mineral oil is light mineral oil. In a particular embodiment, the vegetable oil is cottonseed oil.

The pharmaceutically acceptable carrier may alternately or in addition comprise a suitable oil replacement. Oil replacements include alkanes having at least 10 carbon (e.g., isohexadecane), benzoate esters, aliphatic esters, noncomodogenic esters, volatile silicone compounds (e.g., cyclomethicone), and volatile silicone substitutes. Examples of benzoate esters include C₁₂C₁₅ alkyl benzoate, isostearyl benzoate, 2-ethyl hexyl benzoate, dipropylene glycol benzoate, octyldodecyl benzoate, stearyl benzoate, and behenyl benzoate. Examples of aliphatic esters include C₁₂C₁₅ alkyl octonoate and dioctyl maleate. Examples of noncomodogenic esters include isononyl isononanoate, isodecyl isononanoate, diisostearyl dimer dilinoleate, arachidyl propionate, and isotridecyl isononanoate. Examples of volatile silicone substitutes include isohexyl decanoate, octyl isononanoate, isononyl octanoate, and diethylene glycol dioctanoate.

Cyclomethicone is an evaporative silicone which may be included in the carrier to assist in making the composition amenable to ejection from a spray dispenser. Furthermore, due to its evaporative property, cyclomethicone may assist in retaining and fixing the formulation on the surface to which it is sprayed e.g. a wound site.

The carrier may further comprise at least one wax. Waxes include for example, beeswax; vegetable waxes, sugar cane waxes, mineral waxes, and synthetic waxes. Vegetable waxes include for example, carnauba, candelilla, ouricury and jojoba wax. Mineral waxes include for example, paraffin wax, lignite wax, microcrystalline waxes and ozokerites. Synthetic waxes include for example, polyethylene waxes.

The pharmaceutical composition may be formulated in any of a number of forms such as for example, a capsule (including a softgel capsule or a hard gelatin capsule), a tablet, a gel, a liposome, a suppository, a suspension, an ointment, a solution, an emulsion or microemulsion, a film, a cement, a powder, a glue, an aerosol, a spray and a gel.

For preparing the pharmaceutical composition, the isolated fraction of a Cupressaceae resin may be suitably formulated as inclusion complexes, nanoemulsions, microemulsions, powders and liposomes. In a particular embodiment, an inclusion complex comprises at least one cyclodextrin. In a particular embodiment, cyclodextrins comprise hydroxypropyl-β-cyclodextrin. In a particular embodiment, nanoemulsions comprise droplets having average particle size of less than 800 nm. In a particular embodiment, the droplets have average particle size of less than 500 nm. In a particular embodiment, the droplets have average particle size of less than 200 nm. In a particular embodiment, powders are spray dried powders. In a particular embodiment, liposomes comprise multilamellar vesicles. In a particular embodiment, a microemulsion comprises a non-ionic surfactant. Non-ionic surfactants include, without limitation, polyoxyl castor oils, polyoxyethylene sorbitan fatty acid esters (polysorbates), a poloxamer, a vitamin E derivative, polyoxyethylene alkyl ethers, polyoxyethylene sterates, saturated polyglycolyzed glycerides or combinations thereof.

Various formulations of isolated fractions of Cupressaceae plant material and preparation thereof are disclosed herein in Examples. The pharmaceutical compositions of the invention may be administered by any means that achieve their intended purpose. For example, administration may be by oral, parenteral, topical or transdermal routes. Parenteral administration includes intravenous, intramuscular, subcutaneous, intradermal, intraperitoneal, intraarterial, intrauterine, intraurethral, intracardial, intracerebral, intracerebroventricular, intrarenal, intrahepatic, intratendon, intraosseus and intrathecal routes of administration. Topical administration includes application via a route selected from dermal, vaginal, rectal, inhalation, intranasal, ocular, auricular and buccal., The administering may in addition comprise a technique or means such as electroporation, or sonication in order to assist in their delivery, for example transdermally. Other techniques which may be employed include for example, radio frequency or pressurized spray application.

The dosage administered will be dependent upon the age, health, and weight of the subject, the use of concurrent treatment, if any, frequency of treatment, and the nature of the effect desired. The amount of the isolated fraction of the present invention in any unit dosage form comprises a therapeutically effective amount which may vary depending on the recipient subject, route and frequency of administration.

In general, the amount of the isolated Cupressaceae resin fraction present in the pharmaceutical composition may conveniently be in the range from about 0.01% to about 25%, such as 0.01% to about 12%, on a weight per weight basis, based on the total weight of the composition. For topical use, the percentage of an isolated Cupressaceae resin fraction in the composition may be in the range from about 0.05% to about 10%. For administration by injection, the percentage of an isolated Cupressaceae resin fraction in the composition may be conveniently in the range from about 0.1% to about 7%. For oral administration, the percentage of an isolated Cupressaceae resin fraction in the composition may be in the range from about 0.005% to about 7%.

The pharmaceutical compositions of the invention may be manufactured in a manner which is itself known to one skilled in the art, for example, by means of conventional mixing, granulating, dragee-making, softgel encapsulation, dissolving, extracting, or lyophilizing processes. Thus, pharmaceutical compositions for oral use may be obtained by combining the active compounds with solid and semi-solid excipients and suitable preservatives, and/or antioxidants. Optionally, the resulting mixture may be ground and processed. The resulting mixture of granules may be used, after adding suitable auxiliaries, if necessary, to obtain tablets, softgels, capsules, or dragee cores.

Suitable excipients are, in particular, fillers such as saccharides, e.g., lactose or sucrose, mannitol or sorbitol; cellulose preparations and/or calcium phosphates, e.g., tricalcium phosphate or calcium hydrogen phosphate; as well as binders, such as starch paste, using, e.g., maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are flow-regulating agents and lubricants, e.g., silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations, such as acetylcellulose phthalate or hydroxypropymethyl-cellulose phthalate, are used. Dye stuffs or pigments may be added to the tablets or dragee coatings, e.g., for identification or in order to characterize combinations of active compound doses.

Other pharmaceutical compositions for oral use include push-fit capsules made of gelatin, as well as soft, sealed capsules made of gelatin and a plasticizer, such as glycerol or sorbitol. The push-fit capsules can contain the active compounds in the form of granules, which may be mixed with fillers, such as lactose; binders, such as starches; and/or lubricants, such as talc or magnesium stearate and, optionally, stabilizers. In soft capsules, the active compounds are preferably dissolved or suspended in suitable liquids, such as fatty oils, or liquid paraffin. In addition, stabilizers may be added.

Other pharmaceutical compositions for oral use include a film designed to adhere to the oral mucosa, as disclosed for example in U.S. Pat. Nos. 4,713,243; 5,948,430; 6,177,096; 6,284,264; 6,592,887, and 6,709,671.

Pharmaceutical compositions in the form of suppositories consist of a combination of the active compound(s) with a suppository base. Suitable suppository bases include for example, natural or synthetic triglycerides, polyethylene glycols, or paraffin hydrocarbons.

Formulations for parenteral administration include suspensions and microparticle dispersions of the active compounds as appropriate. In a particular embodiment, oily injection suspensions may be administered. Suitable lipophilic solvents or vehicles include fatty oils, e.g., sesame oil, or synthetic fatty acid esters, e.g., ethyl oleate, triglycerides, polyethylene glycol-400, cremophor, or cyclodextrins. Injection suspensions may contain substances which increase the viscosity of the suspension include, e.g., sodium carboxymethyl cellulose, sorbitol, and/or dextran. Optionally, the suspension may also contain stabilizers.

Pharmaceutical compositions can also be prepared using liposomes comprising the active ingredient. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolisable lipid capable of forming liposomes can be used. In general, the preferred lipids are phospholipids and the phosphatidyl cholines (lecithins), both natural and synthetic. Methods to form liposomes are known in the art, as disclosed for example, in Prescott, Ed., Methods in Cell Biology, Volume XIV, Academic Press, New York, N.Y. (1976) and in U.S. Pat. No. 7,048,943.

Formulations for topical administration include ointments. Suitable carriers include vegetable or mineral oils, white petrolatum, branched chain fats or oils, animal fats and waxes. The preferred carriers are those in which the active ingredient is soluble. Stabilizers, humectants and antioxidants may also be included, as well as agents imparting color or fragrance, if desired. Ointments may be formulated for example, by mixing a solution of the active ingredient in a vegetable oil such as almond oil with warm soft paraffin, and allowing the mixture to cool.

The pharmaceutical composition may comprise an oil-in-water emulsion or microemulsion in order to facilitate its formulation for oral, parenteral or topical use Such emulsions/microemulsions generally include lipids, surfactants, optionally humectants, and water. Suitable lipids include those generally know to be useful for creating oil-in-water emulsions/microemulsions, for example fatty acid glyceride esters. Suitable surfactants include those generally known to be useful for creating oil-in-water emulsions/microemulsions wherein lipids are used as the oil component in the emulsion. Non-ionic surfactants may be preferred, such as for example, ethoxylated castor oil, phospholipids, and block copolymers of ethylene oxide and propylene oxide. Suitable humectants, if used, include for example propylene glycol or polyethylene glycol.

The pharmaceutical composition may be formulated in the form of a gel, such as a hydrogel formed from a gel-forming polymer such as carrageenan, xanthan gum, gum karaya, gum acacia, locust bean gum, guar gum. A hydrogel may be combined with an oil-in-water emulsion comprising the active ingredient.

The pharmaceutical composition may be formulated in the form of a cement such as those comprising polymethylmetacrylate (PMMA) or calcium phosphate, as are used in orthopedic surgery.

The pharmaceutical composition may be formulated in the form of a powder, in particular such as those used for transdermal applications using radio frequency, as described for example, in U.S. Pat. Nos. 6,074,688 and 6,319,541 and WO 2006/003659.

The pharmaceutical composition may be formulated in the form of a glue, such as those comprising octocyanoacrylate used for wound closure applications.

### Therapeutic Uses

The present invention provides therapeutic uses and methods for treating impaired neurological function, preventing or treating fibrotic conditions, preventing or treating surgical adhesions, reducing scar formation at a wound site, preventing or treating gliosis, and inducing or promoting tissue repair following an injury or insult in a subject in need thereof.

The isolated fractions and/or compositions are administered to a subject in a therapeutically effective amount. According to certain embodiments, the step of administering the compositions may comprise any acceptable route including oral, topical, parenteral, and transdermal. Parenteral administration includes intravenous, intramuscular, subcutaneous, intradermal, intraperitoneal, intraarterial, intrauterine, intraurethral, intracardial, intracerebral, intracerebroventricular, intrarenal, intrahepatic, intratendon, intraosseus and intrathecal routes of administration. Topical administration includes application via a route selected from dermal, vaginal, rectal, inhalation, intranasal, ocular, auricular and buccal. Each possibility is a separate embodiment.

In particular embodiments, the step of administering comprises contacting cells of a particular type, of a particular lineage or at a particular stage of differentiation, with the composition. The cells may be any of a wide variety of cell types, including in particular, neural cells, neuronal cells, endothelial cells, epithelial cells and stem cells of said lineages. Further, the cells may be of any lineage for example, ectodermal, mesodermal, entodermal lineages and stem cells of said lineages. In various embodiments, the step of contacting cells is carried out in vivo, ex vivo or in vitro.

The uses and methods disclosed herein for treating impaired neurological function are particularly advantageous for subjects afflicted with neurodegenerative conditions and diseases, including in particular, vascular dementia, senile dementia, Alzheimer's disease, amyotrophic laterial sclerosis (ALS), multiple sclerosis, and Parkinson's disease. In other cases, the subject may be suffering from impaired neurological function due to trauma or stroke. As used herein, stroke includes any of cerebral ischemia, subarachnoid hemorrhage and intracerebral hemorrhage. The impaired neurological function may further be due to exposure to a drug, such as an anesthetic. Each possibility is a separate embodiment.

The invention further provides therapeutic methods and use of the fractions and composition for preventing or treating a fibrotic condition. Fibrotic conditions may arise as a consequence of infections, autoimmune diseases, physical injuries, metabolic disorders, exposure to ionizing radiation. In all of these conditions there is a loss of functional tissues and cells, which are replaced by non-functional fibrotic tissue.

Fibrotic conditions include various forms of fibrosis, for example, arterial fibrosis, arthrofibrosis, bladder fibrosis, breast fibrosis, cardiac fibrosis, endomyocardial fibrosis, liver fibrosis, lymph node fibrosis, mediastinal fibrosis, muscle fibrosis, myelofibrosis, nephrogenic systemic fibrosis, pancreatic fibrosis, pleural fibrosis progressive massive fibrosis, pulmonary fibrosis, renal fibrosis, retroperitoneal fibrosis, skin fibrosis, thyroid fibrosis, cirrhosis, vascular stenosis, restenosis, and chronic obstructive pulmonary disease (COPD).

The fibrotic condition may further be selected from scleroderma, a fibromatosis and hypertrophic scarring. Preferred examples of hypertrophic scarring include post-injury scarring and keloid scar.

The invention may further be used for preventing or reducing scar formation at a wound site and for scar-less repair of wounds, such as that sustained from piercing procedures, tattoo procedures and surgical incisions. Other examples of wounds for which the invention may be used to prevent or reduce scarring include burns, amputation wounds, split-skin donor grafts, skin graft donor sites, medical device implantation sites, bite wounds, frostbite wounds, puncture wounds, and shrapnel wounds.

The efficacy of the invention in preventing or reducing scar formation may be assessed by various scar assessment scales, as are known in the art (see for example, (Baryza et al., The Vancouver Scar Scale: an administration tool and its interrater reliability, J Burn Care Rehabil. 1995 September-October; 16(5):535-8; Draaijers et al., The Patient and Observer Scar Assessment Scale: A Reliable and Feasible Tool for Scar Evaluation, Plast Reconstr Surg. 2004 June; 113(7):1960-5). Such scales typically are based on grading of parameters such as vascularization, scar thickness, pigmentation, relief, and the itching experienced by the patient.

The invention may further be used for preventing or treating a surgical adhesion.

Pelvic and abdominal surgical procedures, including hernia repair, gynecological surgeries, and colorectal surgeries, often cause tissue injury that can lead to the formation of post-surgical adhesions in more than 50% of patients. Such injuries include mechanical trauma from retractors and tissue handling, ischemia at suture sites and after electrocautery use, insertion of foreign bodies, tissue desiccation and infection. Adhesion formation can occur in both open and laparoscopic approaches, and may be a common cause of small bowel obstruction, pelvic pain, and female infertility (for adhesions involving the ovaries or fallopian tubes).

The efficacy of the present invention in reducing or preventing surgical adhesions may be assessed using a small animal model, in which surgical injury is mimicked by application of abrasion or electrocautery to various organs. Such models are known in the art, and may employ for example rats (Golan et al., (1995) Hum. Reprod., 10, 1797-1800); mice (Haney and Doty, (1992) Fertil. Steril. 57, 202-208); rabbits (Marana et al., (1997) Hum. Reprod., 12, 1935-1938); or pigs (Moritz et al., (1993) Gynecol. Oncol., 48, 76-79).

The invention may further be used for preventing or reducing gliosis, in particular that associated with anoxic injury. The gliosis may be associated with a neurodegenerative disorder selected from Alzheimer's disease, Korsakoffs syndrome, multiple system atrophy, prion disease, multiple sclerosis, AIDS dementia complex, Parkinson's disease, ALS and Huntington's disease.

Further disclosed herein are uses of Cupressaceae fractions and compositions and therapeutic methods for inducing or promoting tissue regeneration in subjects who have tissue damage, for example, tissue damage associated with, or sustained as a result of an injury or insult, such as a myocardial infarction, a pulmonary embolism, a cerebral infarction, peripheral artery occlusive disease, a hernia, a splenic infarction, a venous ulcer, an axotomy, a retinal detachment or a surgical procedure.

The Cupressaceae fractions and compositions of the invention may be contacted with a tissue or organ to be treated, using a means selected from the group consisting of electroporation, sonication, radio frequency, pressurized spray and combinations thereof.

The step of contacting may comprise establishing contact between interstitial fluid and the composition to be used. This may be particularly advantageous for wounds which are surrounded by interstitial fluid. Contact between interstitial fluid and the composition may be accomplished by piercing and/or teasing the dermis with a needle, a microneedle, or an apparatus comprising a plurality of needles or microneedles. Such needles or microneedles are preferably non-hollow and may be fashioned in a plurality for example, on a comb or brush-like apparatus.

The invention may be used for inducing or promoting life span extension in animals.

The invention is suitable for application in humans, non-human mammals, fish and birds.

### Articles of Manufacture

The invention may encompass use of an article of manufacture which incorporates a composition comprising an isolated fraction of Cupressaceae described herein.

The pharmaceutical composition may be in the form of a coating on the article of manufacture, or may be contained within a vessel which is integral to the article of manufacture. The pharmaceutical composition is advantageously present as a coating on devices which are inserted to the body and are intended for integration therein, for example an implant.

The pharmaceutical composition may be advantageously incorporated onto or into articles used in wound healing or tissue repair, for example, a dressing or bandage.

In other cases, the pharmaceutical composition may be incorporated to a delivery device such as a needle, an injection device or a spray dispenser from which the composition is delivered to a body site requiring therapy, for example a wound site.

Articles of manufacture include, but are not limited to a fabric article, a diaper, a wound dressing, a medical device, a needle, a microneedle, an injection device and a spray dispenser. In a particular embodiment, the article of manufacture comprises a plurality of microneedles.

Medical devices include, but are not limited to a prosthetic, an artificial organ or component thereof, a valve, a catheter, a tube, a stent, an artificial membrane, a pacemaker, a sensor, an endoscope, an imaging device, a pump, a wire and an implant. Implants include, but are not limited to a cardiac implant, a cochlear implant, a corneal implant, a cranial implant, a dental implant, a maxillofacial implant, an organ implant, an orthopedic implant, a vascular implant, an intraarticular implant and a breast implant.

In a particular embodiment, the medical device is an organ implant, which may in certain cases comprise autologous cells of the subject.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

### Example 1

### Preparation of Isolated Fractions from Cupressus sempervirens Resin

Resin (6.81 grams) from Cupressus sempervirens was combined with absolute ethanol (200 mL) in a 2 L round bottom flask and the mixture was left to stand for 1 hour. Subsequently it was shaken for 15 minutes at 150 rpm on an orbital shaker. The mixture was allowed to stand for 1 hour in order to facilitate precipitation of insoluble material. The ethanol fraction was carefully decanted into a clean pre-weighed 500 mL round bottom flask and the ethanol was evaporated using a rotary evaporator. Hexane (300 mL) was added to the remaining material and the mixture was shaken at 150 rpm on an orbital shaker for 2 hours. Subsequently the flask was allowed to stand overnight in the dark. This yielded a clear yellow-brownish solution with a brown insoluble material on the sides of the flask. The hexane solution was decanted carefully into a clean 1 L round bottom flask and the hexane was removed using a rotary evaporator. This yielded 2.56 g of material (termed “RPh-CY-DS”) having the appearance of a sticky yellow solid/foam.

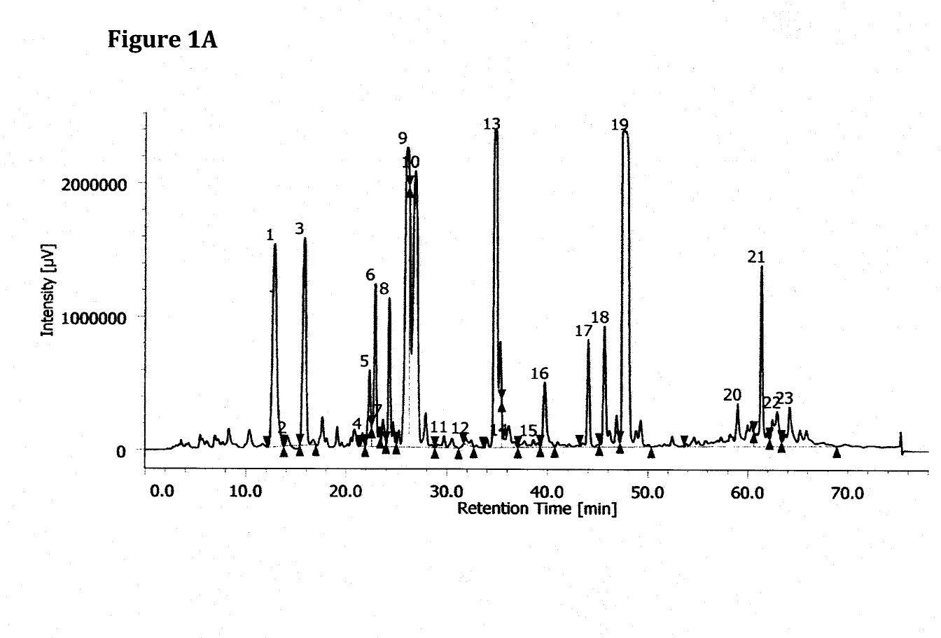

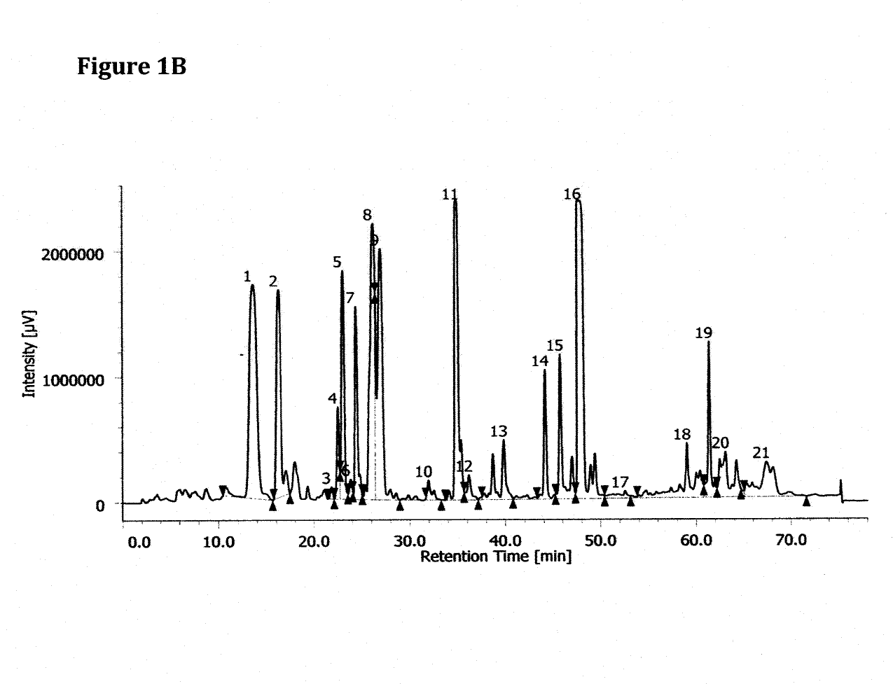

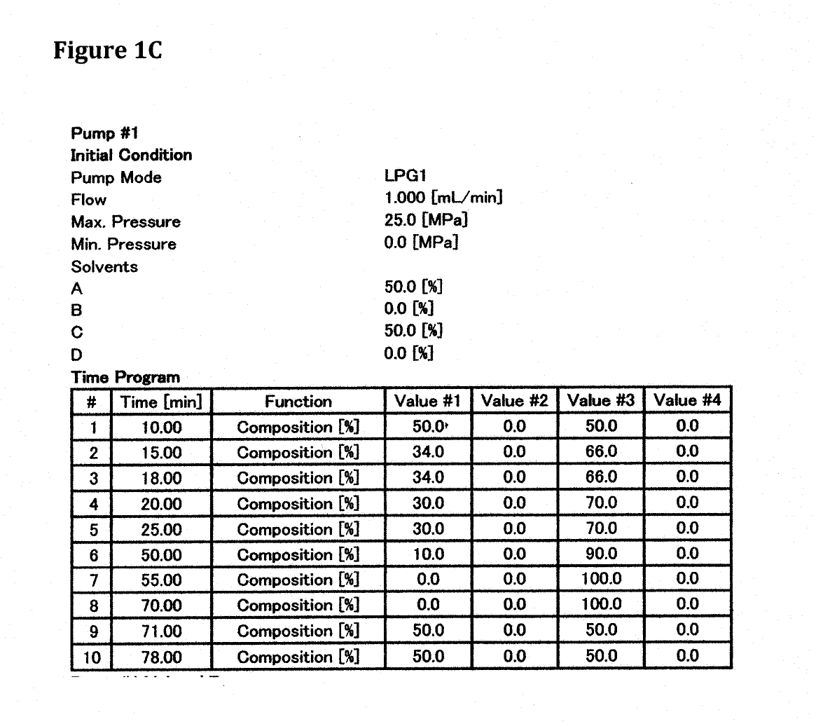

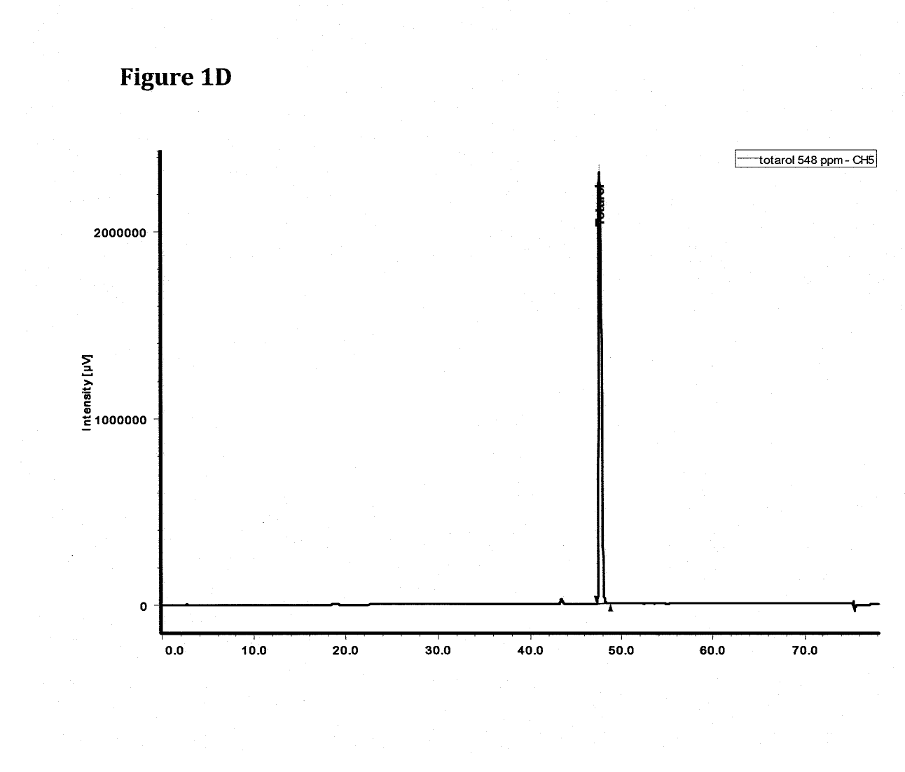

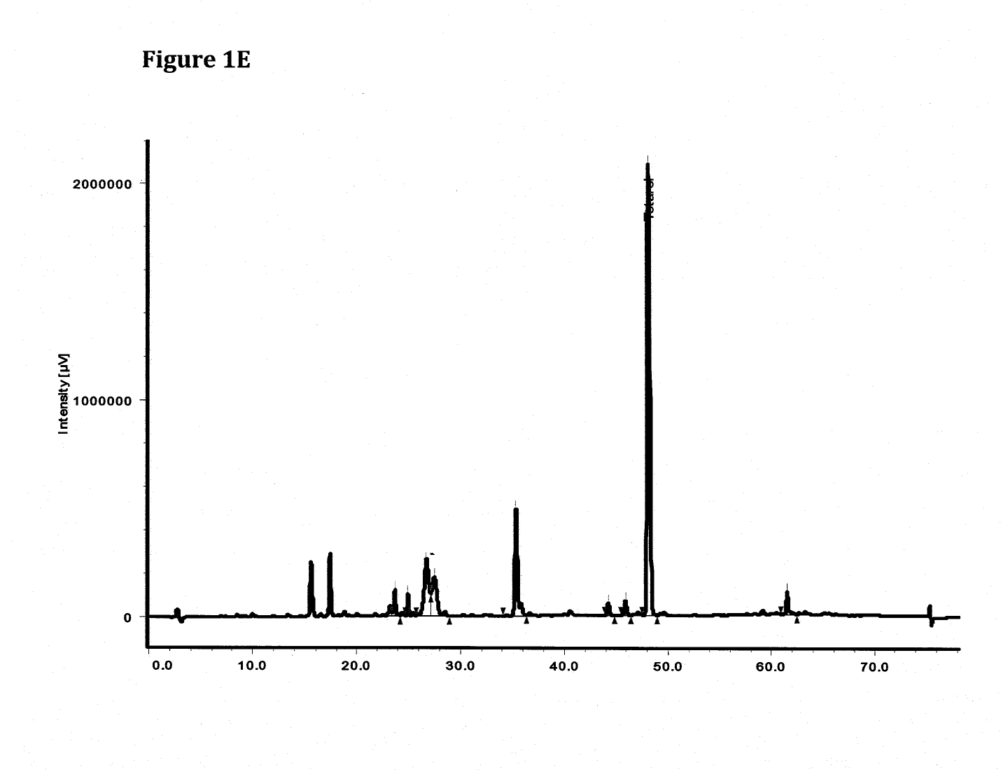

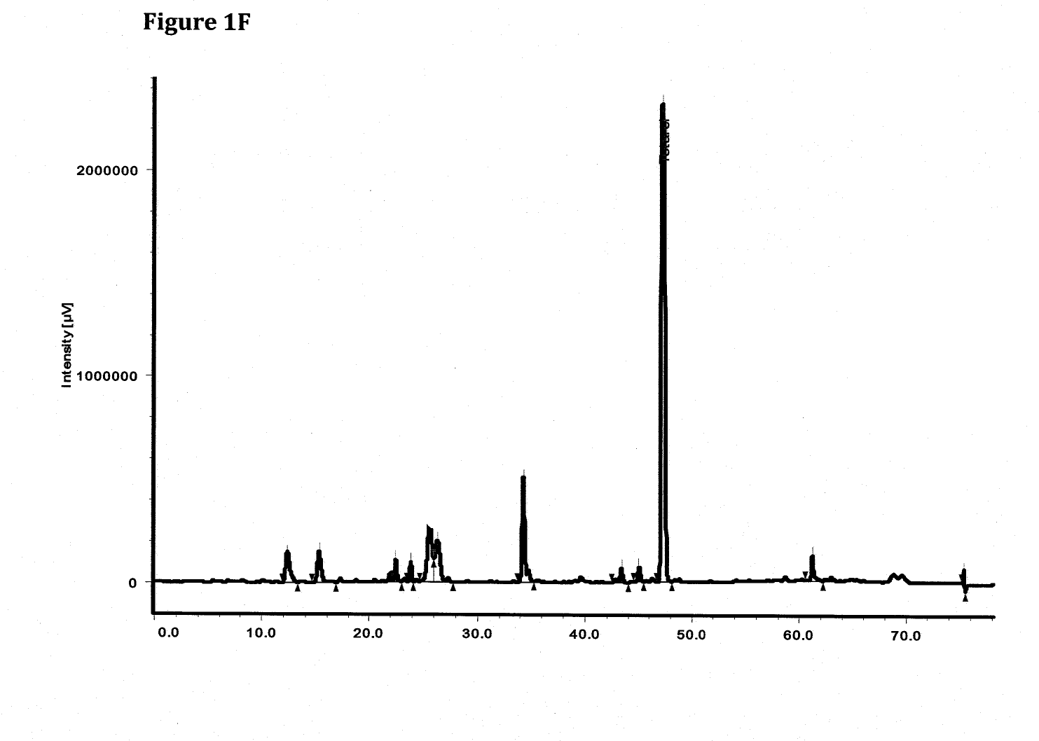

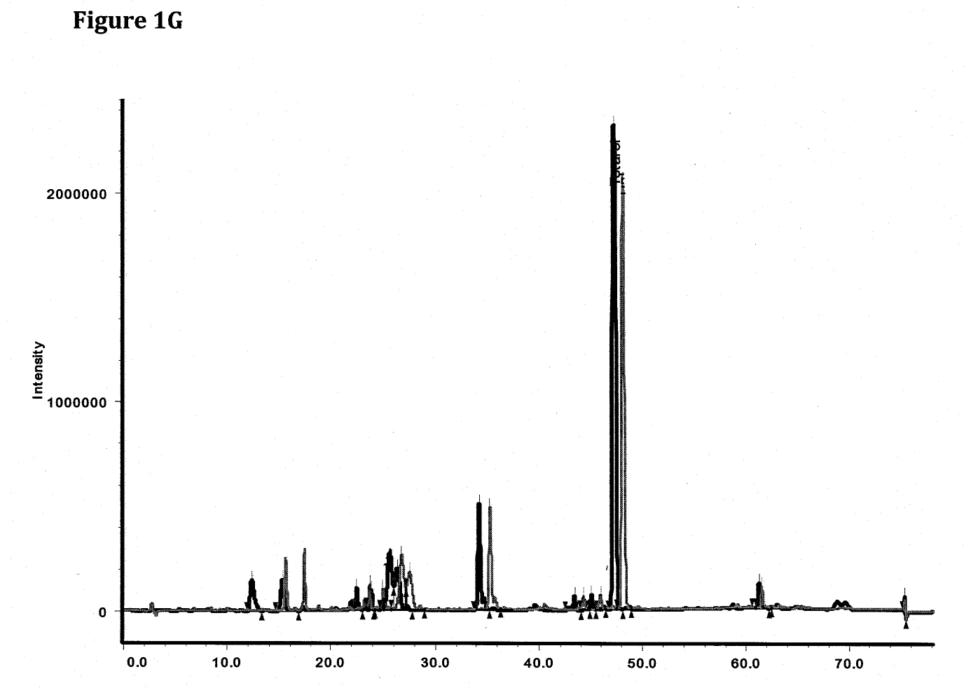

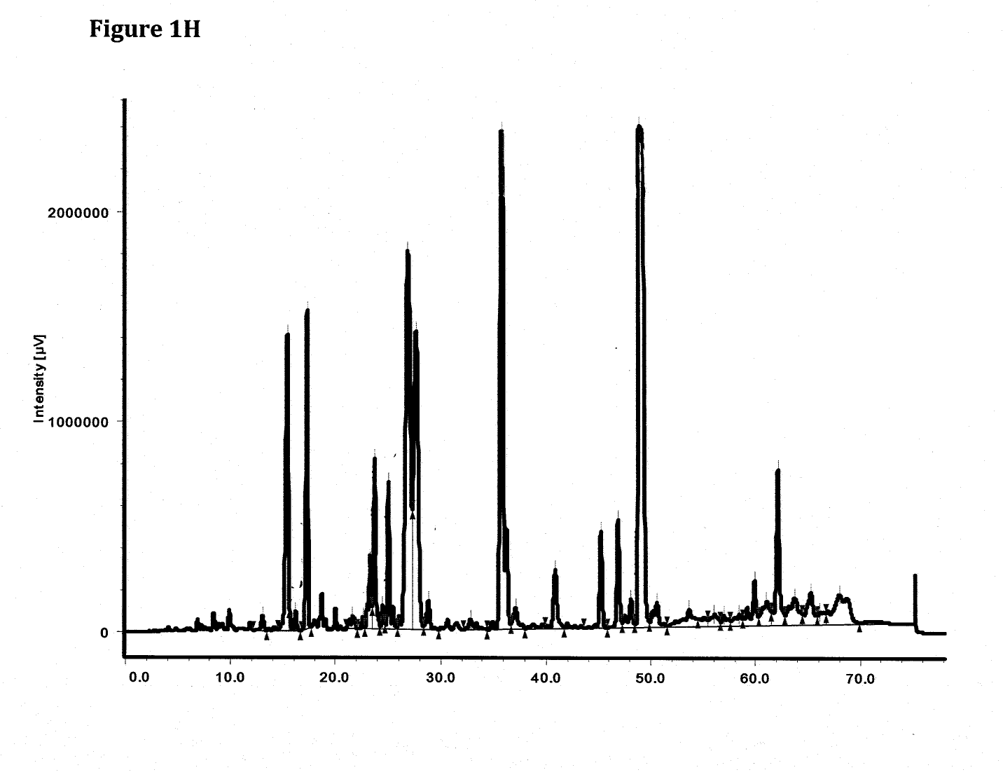

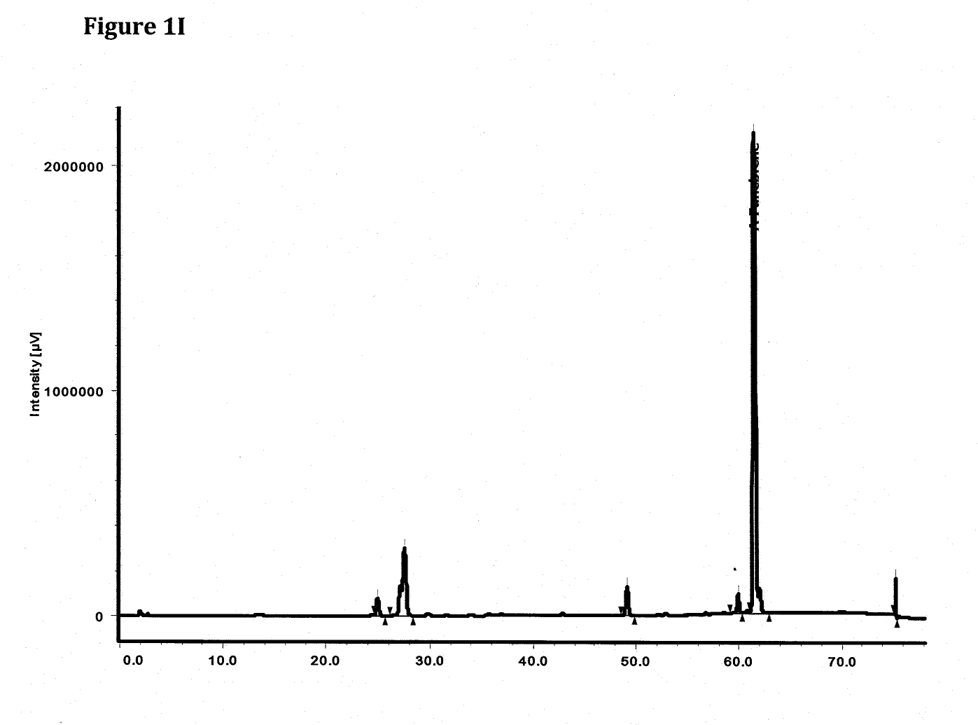

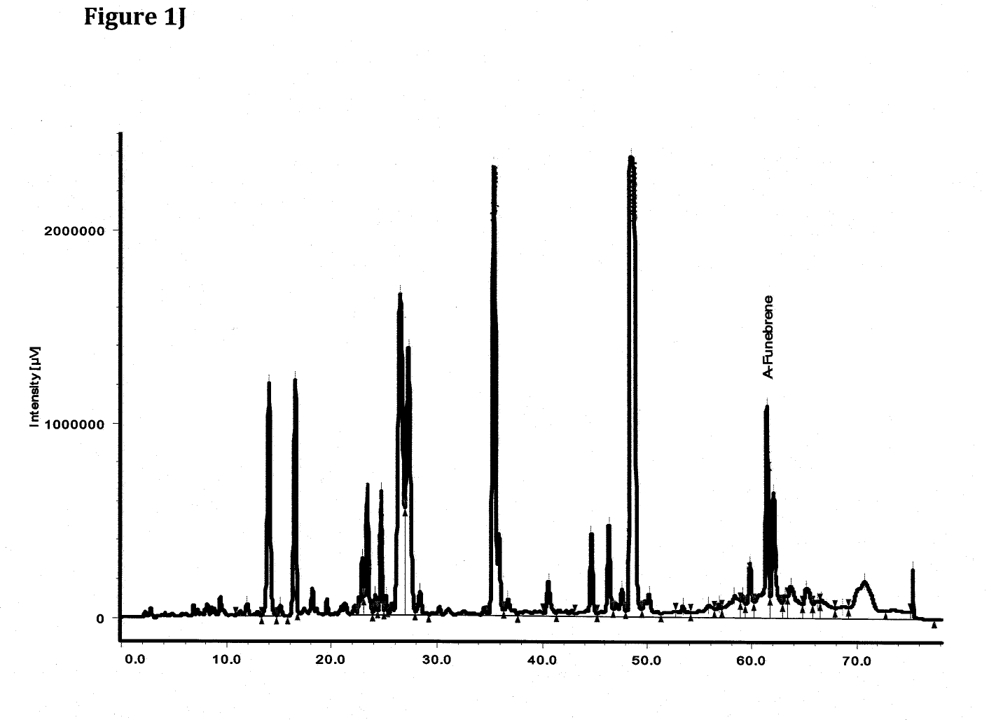

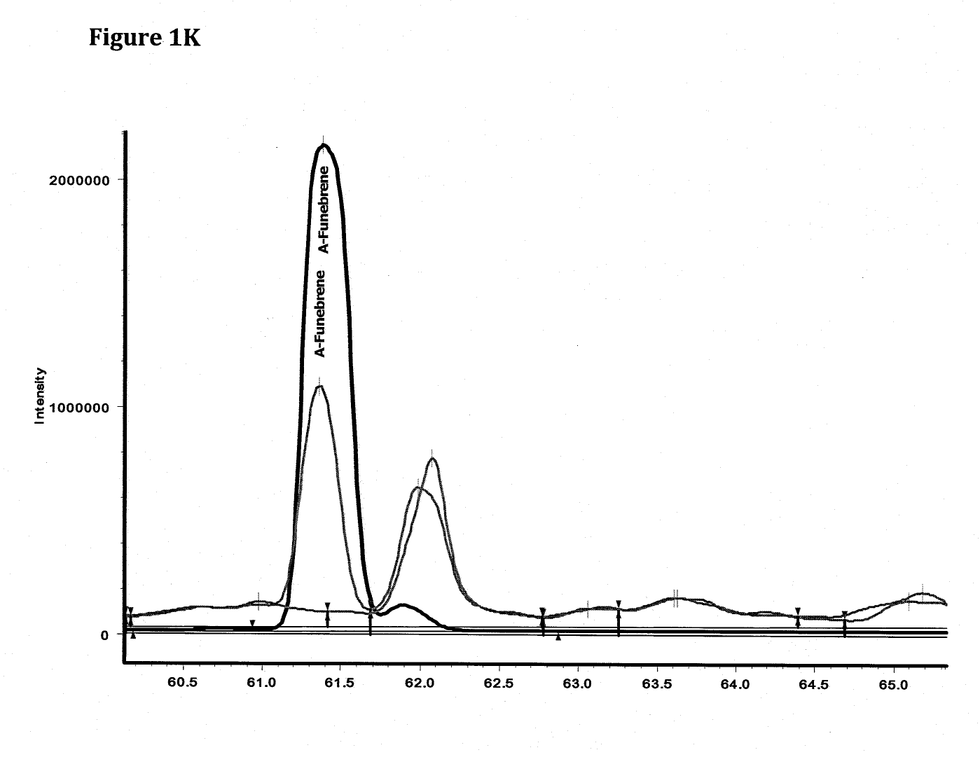

Two batches of RPh-CY-DS were prepared as described above from different individual trees growing in the Carmel region of northern Israel. Analysis by HPLC showed a very similar chemical constitution of the two batches as shown in FIGS. 1A and 1B. Notably, the peaks numbered 11, 13 and 16 in FIG. 1B show absorption at approximately 280 nm which is highly indicative of the presence of an aromatic group, such as those found in the compounds sempervirol, totarol and ferruginol, suggesting the presence of at least some of these compounds, or a combination thereof in the isolated fractions.

Peak number 16 was confirmed to correspond to the compound totarol, following analysis of a preparation of RPh-CY-DS that was diluted and “spiked” with a commercial sample of totarol (Sigma-Aldrich), as demonstrated in FIGS. 1D-1G.

Further comparison with commercial analytical standards showed the absence of a-funebrene in RPh-CY-DS (FIGS. 1H-1K).

### Example 2

### Preparation of a 10% (w/w) Formulation of an Isolated Fraction of Cupressus sempervirens

RPh-CY-DS (5 gram) prepared as described in Example 1, was combined with cottonseed oil (45 gram) in a 250 mL round bottom flask. The mixture was shaken at 150 rpm on an orbital shaker until a clear and homogeneous solution was obtained (2-3 hours). This solution was again subjected to vacuum on a rotary evaporator in order to further remove any residual hexane to give 49.8 gram of a formulation termed “RPh-CY”.

### Example 3

### Preparation of an Isolated Fraction from Sandarac Gum from Tetraclinis articulata

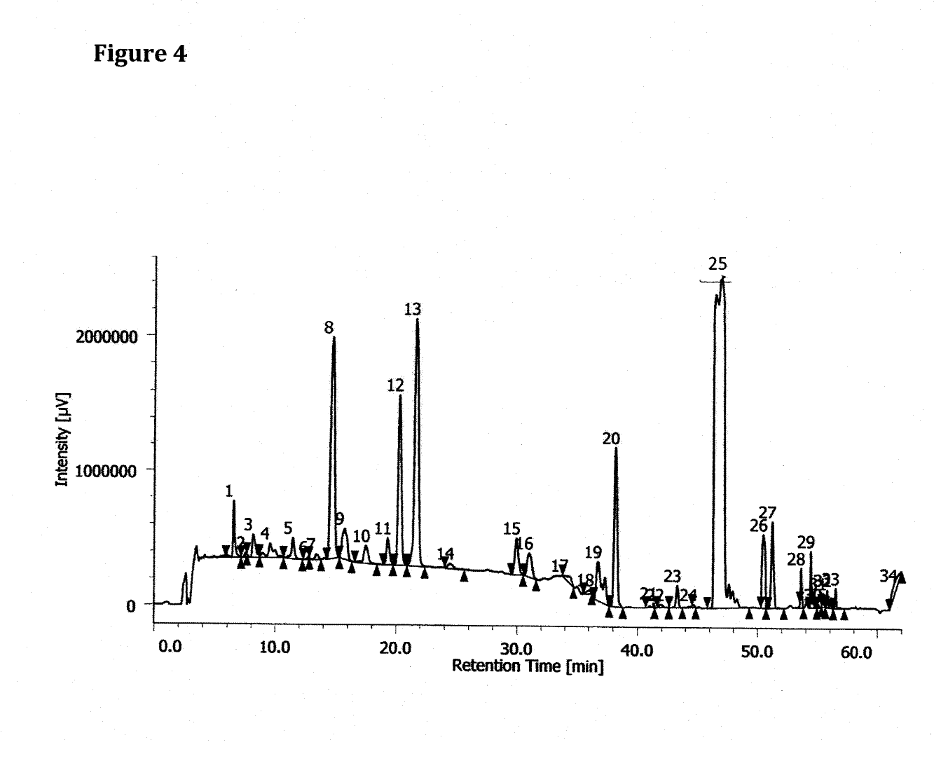

Sandarac gum (10.2 gram) from Tetraclinis articulata was dissolved in absolute ethanol (150 mL) by shaking for 1 hour at 150 rpm in a 250 mL Erlenmeyer flask. The ethanol was decanted carefully from any insoluble material into a 500 mL round bottom flask and the ethanol was evaporated. To the remainder was added hexane (200 mL) and the flask was shaken for 2 hours at 180 rpm on an orbital shaker. Subsequently the flask was left to stand overnight in the dark to allow complete precipitation of insoluble material. The clear hexane solution was carefully decanted into a clean 500 mL round bottom flask and evaporated using rotary evaporator, yielding 1.71 g of an isolated fraction (termed “RPh-SA-DS”) having the appearance of a light yellow viscous material. Analysis by HPLC gave the chromatogram shown in FIG. 4, in which peaks 13, 15, 26, 27 show absorption at 240-250 nm, suggesting the presence of aromatic compounds such as sandaracopimaric acid and 4-epidehydroabietic acid.

### Analytical HPLC Method Used:

Solvent A: 0.05% Formic acid

### Solvent B: 3% THF in Acetonitrile

### Gradient Program:

| # | Time (Min) | % Solvent A | % Solvent B |
|---|---|---|---|
| 1 | 5.00 | 50 | 50 |
| 2 | 23.00 | 46 | 54 |
| 3 | 30.00 | 40 | 60 |
| 4 | 40.00 | 24 | 76 |
| 5 | 45.00 | 20 | 80 |
| 6 | 48.00 | 20 | 80 |
| 7 | 50.00 | 0 | 100 |
| 8 | 55.00 | 0 | 100 |
| 9 | 56.00 | 50 | 50 |
| 10 | 62 | 50 | 50 |

### Example 4

### Isolation and Characterization of “Peak 25” (FIG. 4) from RPh-SA-DS by Preparative HPLC

A solution of RPh-SA-DA (90 mg/ml in methanol) was subjected to preparative HPLC.

### HPLC Method:

| Wave | | Eluent(Acetonitrile: | |
|---|---|---|---|
| length(nm) | Time (mn) | 0.8% acetic ac in water) | |
| 205 | 30 | 50:50 | Conditioning |
| 205 | 11 | 50:50 | |
| 250 | 52 | 70:30 | peaks |
| 205 | | Me—OH | Wash |

“Peak 25” was found to consist of a mixture of E- and Z-communic acid (E:Z≈3:1). The structures were confirmed by comparison of the ¹H-NMR and ¹³C-NMR spectra with literature data. (Olate et al. Molecules, 2011, 16, p. 10653-10667).

### Example 5

### Preparation of a 10% (w/w) Formulation of the Isolated Mixture of E/Z-Communic Acids (RPh-CMA)

E/Z-communic acid (100 mg) as obtained in Example 4 was dissolved in 900 mg cottonseed oil by shaking for 3 hours.

### Example 6

### Preparation of a 5% (w/w) Formulation of an Isolated Fraction of Tetraclinis articulata

Cottonseed oil (18 g) was added to 2 g RPh-SA-DS, prepared according to Example 3. The mixture was shaken at 150 rpm on an orbital shaker until a clear and homogeneous solution was obtained (2-3 hours). The solution was subjected to vacuum on a rotary evaporator in order to remove any residual hexane to give 20.0 g of a formulation termed “RPh-SA”.

### Example 7

### pH-CY, RPh-SA and RPh-CMA Induce Neuronal-Like Differentiation in Retinal Pigment Epithelial Cell Cultures

### Overview

The present invention is directed to induction of differentiation and cell maturation, and has direct application to regeneration of functional tissue, in particular neuronal tissue. Our experimental findings show that each of RPh-CY, RPh-SA and RPh-CMA induce morphological differentiation of retinal pigment epithelial cells to neuronal cells producing axons, dendrites and junctions between cells known as synapses. The neuronal cell differentiation induced by RPh-CY, RPh-SA and RPh-CMA strongly suggests that these fractions effect neuronal stem cell differentiation into functional neurons.

Current dogma on the pathology of dementia and Alzheimer's disease holds that the deficiency involves the failure of neurons to form functional synaptic junctions (see for example, Kimura R, Ohno M. Impairments in remote memory stabilization precede hippocampal synaptic and cognitive failures in 5XFAD Alzheimer mouse model. Neurobiol Dis. 2008 Nov. 5).

Accordingly, the experiments described herein support use of isolated fractions of Cupressaceae, such as those described in Examples 1 and 3, as a therapeutic modality to elicit neuro-regeneration in neurodegenerative diseases such as dementia and Alzheimer's disease.

### Retinal Pigment Epithelium (RPE) Cells

Studies aimed at evaluating effects of RPh-CY, RPh-SA and RPh-CMA on various cell lines of human origin included investigation of ARPE-19 cells, a non-malignant human retinal pigment epithelial cell line of neuronal origin.

The retinal pigment epithelium (RPE) is a single layer of hexagonal pigmented epithelial cells of neuronal origin, which forms the outermost cell layer of the eye retina and is attached to the underlying choroid. RPE functions include support, nourishment and protection of the underlying photoreceptors of the neuro-retina.

RPE cells are involved in the phagocytosis of the outer segment of photoreceptor cells, in the vitamin A cycle where they isomerize all-trans retinol to 11-cis retinal and in supplying the photoreceptors with D-glucose, amino acids and ascorbic acid.

Although in vivo the RPE is pigmented, ARPE-19 cells do not form melanin and are not pigmented. In culture the cells grow as spindle shaped and as polygonal cells.

### Methods

ARPE-19 cells (obtained from the American Type Culture Collection, ATCC) were plated in flat bottom 96 well tissue culture microplates (Costar) at a concentration of 2-5×10³ cells per well (1-2.5×10⁴ cells/mL) in a growth medium consisting of DMEM:Ham F-12, 1:1, supplemented with 10% Fetal Bovine Serum, 200 mM glutamine, 100 units/mL penicillin and 100 μg/mL streptomycin. The cells were allowed to adhere to the plate surfaces overnight prior to treatment with either RPh-CY or RPh-SA.

RPh-CY and RPh-SA were prepared essentially as described in Examples 2 and 4 respectively, to provide 10% solutions in a carrier composed of grape seed oil, olive oil, cottonseed oil, and Mygliol® (Mygliol® 810 or Mygliol® 812). The preparations were added to the cultures (each having sample medium volume of 200 μl) at volumes of 2 μl, 5 μl, 7.5 μl and 10 μl, corresponding to final fraction concentrations (RPh-CY or RPh-SA) of 0.0125%, 0.05%, 0.125% and 0.5%, respectively. The oil carrier served as a vehicle control and was applied to control cultures at the same volumes.

The cultures were incubated in a 37° C., 5% CO₂ incubator for 48 hr. The medium was then removed, the cultures washed twice with phosphate buffered saline (PBS), fixed with absolute methanol for 10 min and stained with Hemacolor® reagents (Boehringer Mannheim), which stain cells in a manner similar to Giemsa, and may be used in a quantitative cell viability assay (see Keisari, Y. A colorimetric microtiter assay for the quantitation of cytokine activity on adherent cells in tissue culture. J. Immunol. Methods 146, 155-161, 1992).

### Results

Treatment of ARPE-19 RPE cells with RPh-CY, RPh-SA or RPh-CMA was found to induce dramatic morphological changes that are highly suggestive of neurodifferentiation. The morphological changes did not occur in control cultures treated with oil carrier alone, and similar results were seen among the test cultures treated with RPh-CY, RPh-SA or RPh-CMA. The morphological changes were also associated with cessation in cell proliferation, further supporting the conclusion that RPh-CY, RPh-SA and RPh-CMA each induce neuro differentiation.

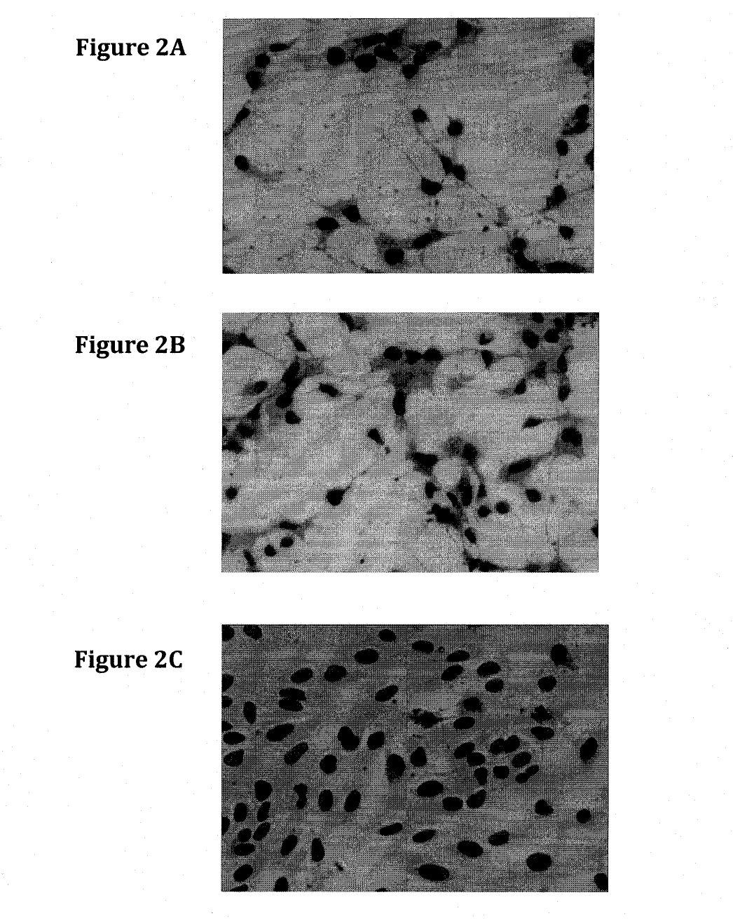

Control oil-treated cultures proliferated and displayed the typical spindle shaped and polygonal growth pattern characteristic of ARPE-19 RPE cells after 48 hours of incubation (FIG. 2C). In contrast, cells treated with RPh-CY (0.25%; 2.5 mg/ml) for the same time period displayed a large number of thin long protrusions, with protrusions in adjacent cells creating a network of inter-connected cells (FIGS. 2A, 2B). This observed pattern suggest that the cells are potentially capable of communicating information amongst one another. Similar networks occur normally between neurons in the central nervous system and enable transmission and processing of information.

In addition, the RPh-CY treated cells rapidly ceased to proliferate and the cells remained in sparse density, supporting the notion of cell differentiation (FIG. 2A, 2B).

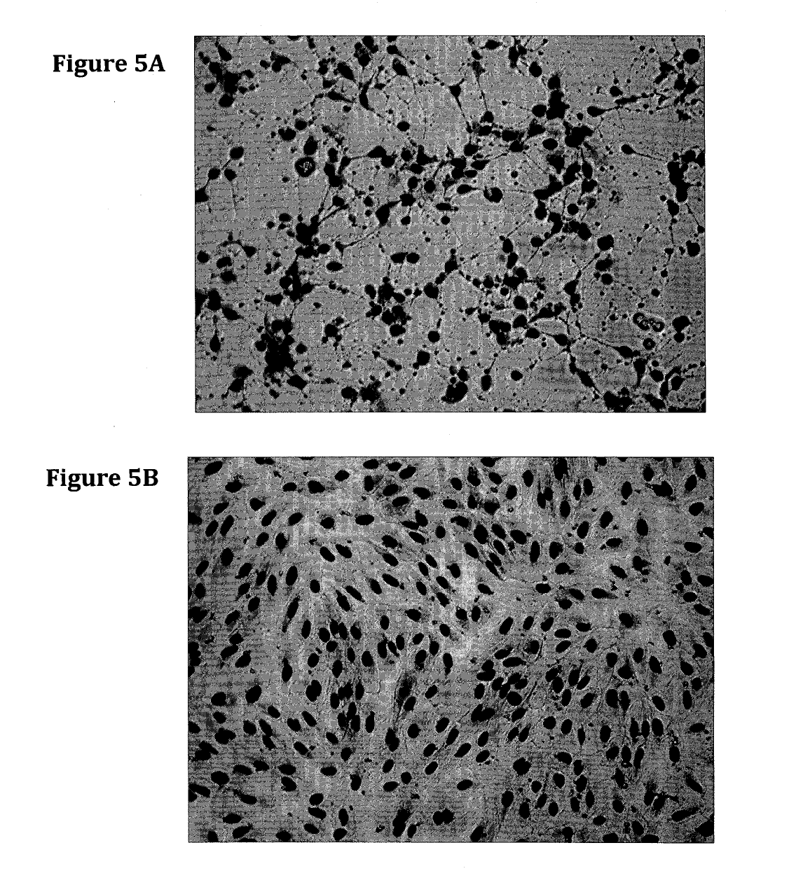

Similarly, ARPE-19 RPE cells treated with RPh-SA exhibited morphological changes that are highly suggestive of neuro-differentiation. As shown in FIG. 5A, cells treated with RPh-SA (0.25%; 2.5 mg/nil; 48 hr incubation) displayed a large number of thin long protrusions with protrusions in adjacent cells creating a network of inter-connected cells. This observed pattern suggest that the cells are potentially capable of communicating information amongst one another. In addition the RPh-SA treated cells rapidly ceased to proliferate and the cells remained in sparse density, further supporting the notion of cell differentiation (FIG. 5A).

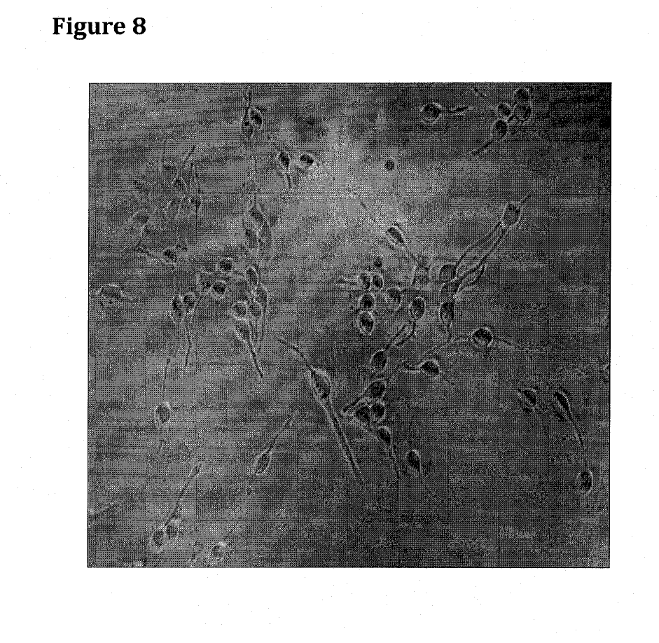

Similarly, ARPE-19 RPE cells treated with RPh-CMA exhibited morphological changes that are highly suggestive of neuro-differentiation. As shown in FIG. 8, cells treated with RPh-SA (0.25%; 2.5 mg/ml; 48 hr incubation) displayed a large number of thin long protrusions with protrusions in adjacent cells creating a network of inter-connected cells. This observed pattern suggest that the cells are potentially capable of communicating information amongst one another. In addition the RPh-SA treated cells rapidly ceased to proliferate and the cells remained in sparse density, further supporting the notion of cell differentiation (FIG. 8).

In contrast, control oil-treated cultures displayed the spindle shaped and polygonal growth pattern characteristic of ARPE-19 RPE cells and underwent proliferation during the 48 hour incubation period (FIG. 5B).

### A Scoring System for the Potency of RPh-CY, RPh-SA and RPh-CMA in Inducing Cell Differentiation

On the basis of the above results, a scoring system was developed to evaluate the potency of Cupressaceae fractions such as RPh-CY, RPh-SA and RPh-CMA, for inducing differentiation in cell culture, with cells plated 2×10³ per well. The grades and their respective descriptions are set out in Table 1.

TABLE 1

| Effect | Grade | | | | | | | | | | | |
|---|---|---|---|---|---|---|---|---|---|---|---|---|
| Proliferation rate | High = 0 | 0 | 0 | 0 | 1 | 1 | 1 | 2 | 1 | 2 | 1 | 2 |
| | Medium = 1 | | | | | | | | | | | |
| | Low = 2 | | | | | | | | | | | |
| Cells form | No = 0 | 0 | 1 | 1 | 1 | 1 | 1 | 1 | 1 | 2 | 2 | 2 |
| elongated | protrusions = 1 | | | | | | | | | | | |
| protrusions | neuron like = 2 | | | | | | | | | | | |
| Neurites | ≦2 = 0 | 0 | 0 | 0 | 0 | 1 | 1 | 1 | 1 | 1 | 4 | 4 |
| (neuron-like | >2 ≦ 3 = 1 | | | | | | | | | | | |
| elongations)/ | >3 = 4 | | | | | | | | | | | |
| body ratio | | | | | | | | | | | | |
| Percent of | ≦10% = 0 | 0 | 0 | 0 | 0 | 0 | 1 | 1 | 2 | 2 | 2 | 3 |
| differentiated | >10% ≦ 30% = 1 | | | | | | | | | | | |
| cells | >30% ≦ 70% = 2 | | | | | | | | | | | |
| | ≧70% = 3 | | | | | | | | | | | |
| Clearly visible | ≦30%, =0 | 0 | 0 | 0 | 0 | 0 | 0 | 0 | 1 | 1 | 1 | 2 |
| junctions between | >30% < 70%, =1 | | | | | | | | | | | |
| neurites and/or | ≧70% = 2 | | | | | | | | | | | |
| cell bodies | | | | | | | | | | | | |
| Visible, clear | <30% = 0 | 0 | 0 | 0 | 0 | 0 | 0 | 0 | 0 | 1 | 1 | 2 |
| synaptic-like | >30% < 50% = 1 | | | | | | | | | | | |
| boutons along the | ≧70% = 2 | | | | | | | | | | | |
| neurites and at the | | | | | | | | | | | | |
| ends of the neurites. | | | | | | | | | | | | |

| | | | | | | | | | | | |
|---|---|---|---|---|---|---|---|---|---|---|---|
| Total Differentiation Grade | 0 | 1 | 1 | 2 | 3 | 4 | 5 | 6 | 10 | 11 | 15 |

| | | | | | | |
|---|---|---|---|---|---|---|
| Differentiation Score | 0 | 1 | 2 | 3 | 4 | 5 |

The RPh-CY, RPh-SA and RPh-CMA formulations prepared as described hereinabove show a Differentiation Grade of at least 11 and a Differentiation Score of at least 4 according to the above outlined scoring system.

### Conclusion

The observed results support the conclusion that the formulations RPh-CY and RPh-SA, corresponding to isolated fractions derived respectively from Cupressus sempervirens and Tetraclinis articulata resins, each display activity in inducing differentiation of ARPE cells into neuronal cells. In addition, the formulation RPh-CMA, containing the mixture of E- and Z-communic acids isolated from RPh-SA-DS also showed activity in inducing differentiation of ARPE cells into neuronal cells.

### Example 8

### Formulations Containing Totarol on its Own Lack Neurodifferentiation Activity

Since peak No. 16 in the HPLC chromatogram of RPh-CY-DS (FIG. 1B) was shown to correspond to the compound totarol, as described in Example 1, formulations consisting only of totarol in cottonseed oil were prepared and analyzed in the neurodifferentiation assay, performed as described in Example 7. Solutions of totarol (Sigma-Aldrich) in cottonseed oil at different concentrations (2-10% w/w) induced no change in ARPE-19 RPE cells, as compared to the vehicle control containing cottonseed oil alone. This strongly suggests that the neurodifferentiation activity induced by RPh-CY is attributable to a combination of compounds contained therein.

### Example 9

### Polar Solvent Extracts of C. sempervirens Lack Neurodifferentiation Activity

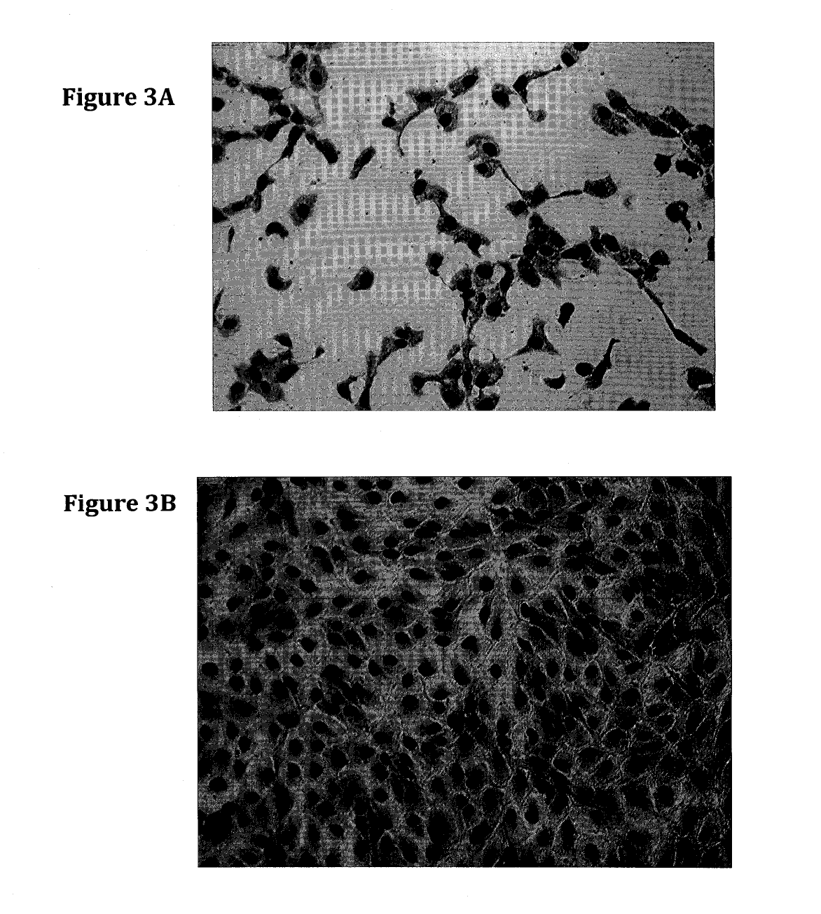

C. sempervirens resin was subjected to the extraction procedure described in Example 1, and the material that was soluble in ethanol but insoluble in hexane, was collected. A sample (1 g) of this material was dissolved in 39 g isopropanol (2.5% w/w). The obtained solution, termed CY-POLAR, was assessed for activity in inducing neurodifferention of ARPE-19 RPE cells, using the methodology described in Example 7. In this experiment, CY-POLAR did not exert any activity in inducing neurodifferentiation of the cells, but rather appeared to induce a state of stress, as shown in FIG. 3A. These results provide evidence that Cupressaceae fractions which are soluble in polar solvents and insoluble in apolar solvents, such as CY-POLAR obtained from C. sempervirens, lack the ability to induce neurodifferentiation.

### Example 10

### Polar Solvent Extracts of T. articulata Lack Neurodifferentiation Activity

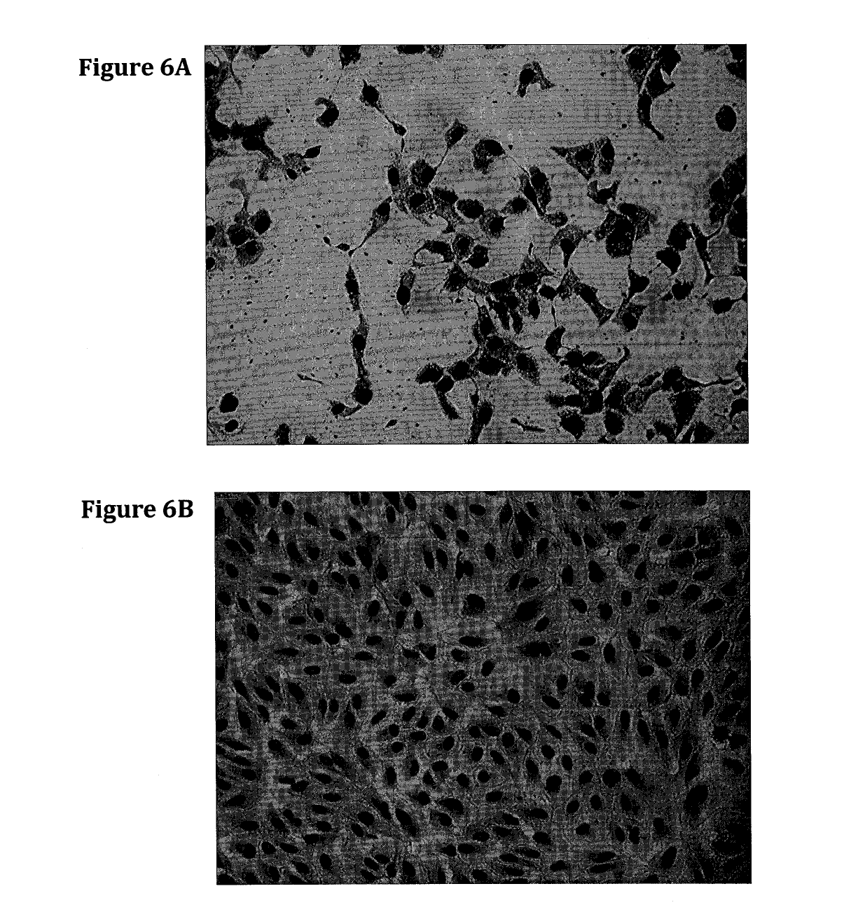

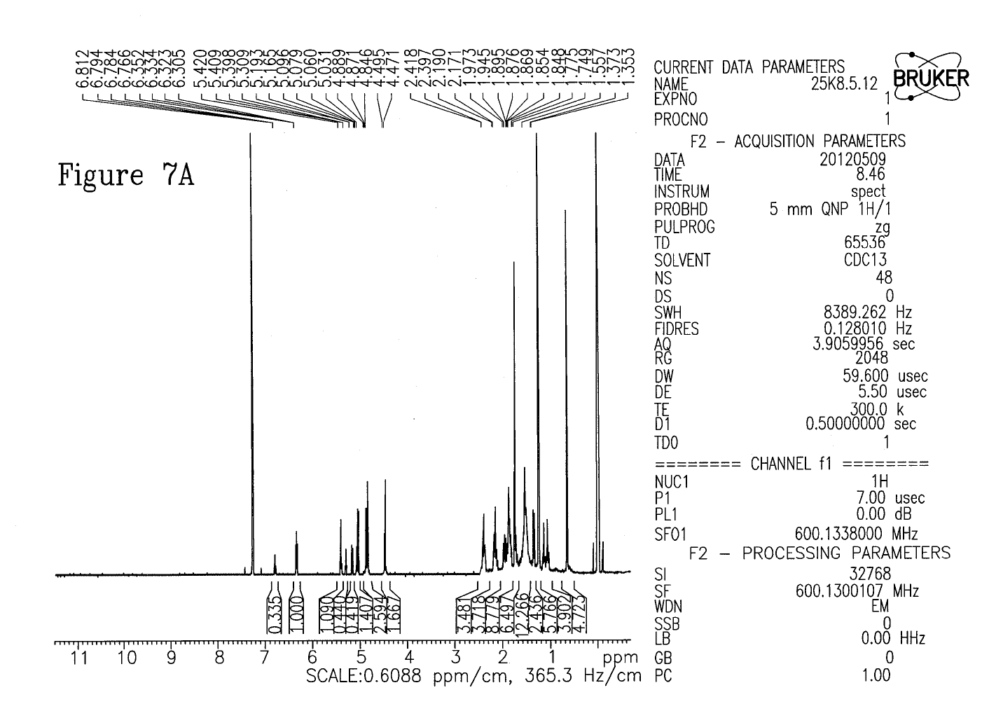

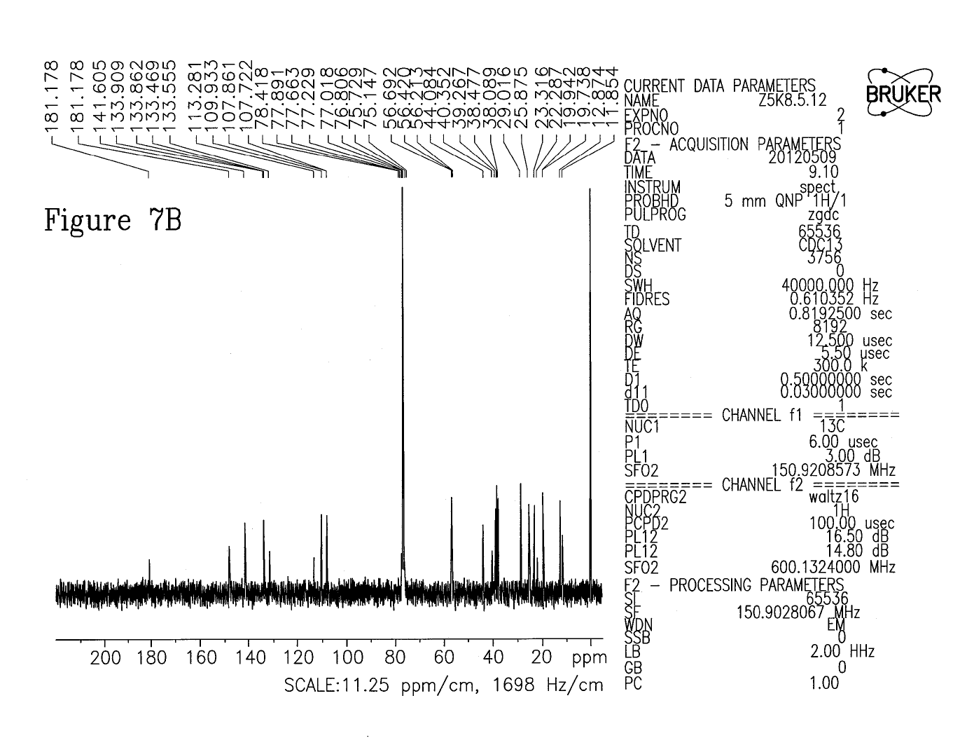

Sandarac gum from Tetraclinis articulata was subjected to the extraction procedure described in Example 3, and the material that was soluble in ethanol but insoluble in hexane, was collected. A sample of this material was dissolved in isopropanol, and the obtained solution, termed “SA-POLAR”, was assessed for it activity in inducing neurodifferention of ARPE-19 RPE cells, using the methodology described in Example 7. In this experiment, SA-POLAR was observed to exert no or negligible activity in inducing neurodifferentiation of the cells, but rather appeared to induce a state of stress, as shown in FIG. 6A. These results provide evidence that Cupressaceae fractions which are soluble in polar solvents and insoluble in apolar solvents, such as SA-POLAR obtained from T. articulata, lack the ability to induce neurodifferentiation.

### Example 11

### Cytotoxicity and Scratch Assay of RPh-CY and RPh-SA Compositions

Scratch Assay

The scratch assay measures the migration rate of fibroblast cells, after a monolayer of cells is scratched so that the layer contains an empty section free of cells. The scratch simulates a wound, wherein closing of the scratch due to migration of the fibroblasts, corresponds to formation of scar tissue. Agents that can inhibit this migratory closure (without exerting cytotoxic effects) have the potential to be used as therapeutic agents for fibroproliferative disorders (for example pulmonary fibrosis) and for use in reduction of scar formation in wound healing.

Methods

Cytotoxicity

3T3 cells (ATCC; CRL-1658) were seeded in a 96 well plate (4×103 cells per well), and 24 hr following cell adherence, cells were treated (in triplicate) with different concentrations (1-50 mg/ml) of the test items, or with the control items cottonseed oil alone, or 10% DMSO, or were left untreated. All treatments were introduced in a final volume of 10 W/well within a total volume of 100 μl/well. Cytotoxicity was determined 48 hr after additional of test items, using the XTT kit (Biological industries, Israel). Raw data was analyzed using Prism4 statistical software following blank reduction. The cottonseed oil vehicle treated control was used to calculate 100% viability and OD % was calculated.

Scratch Assay

3T3 cells (ATCC; CRL-1658) were seeded in a 96 well plate (4×103 cells per well), and 24 hr following cell adherence, scratch induction was performed in each monolayer. Cultures were washed once with complete medium to eliminate detached cells, and then treated (in triplicate) with either RPh-CY (0.125 μg/μl); RPh-SA (0.125 μl/μl); cottonseed oil vehicle alone; FCS 1% (positive control for migration inhibition), or were left untreated (complete medium). All treatments were introduced in a final volume of 10 μl within a total volume of 100 μl per well. Gap closure was monitored by photographing each of the wells at 0 time (T0) following addition of test material and at the time points 24, 48 and 55 hr after addition of treatment material. Photographs were obtained with a Juli microscope (Ornat).

The extent of wound healing was determined from data analysis involving the gap measurement at each time point. The gap width was determined based on 5 width measurements in pixels that were averaged using ImageJ software. To enable collective analysis, the initial gap width (T0) in each well served as the 100% value for the other time points and was used for determination of the average and standard deviation (SD) in each group. Each average point is based on 15 measurements (5 for each replicate).

Results

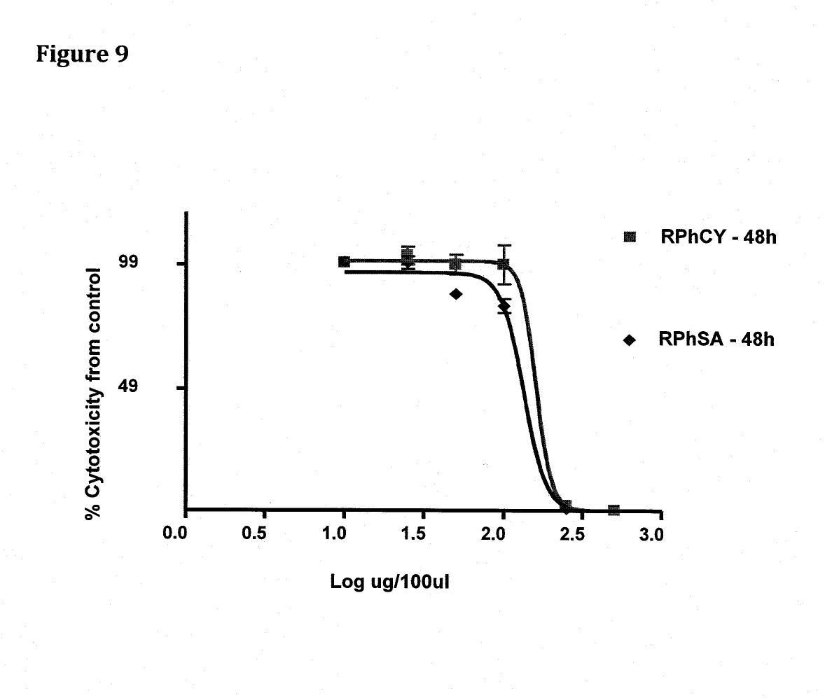

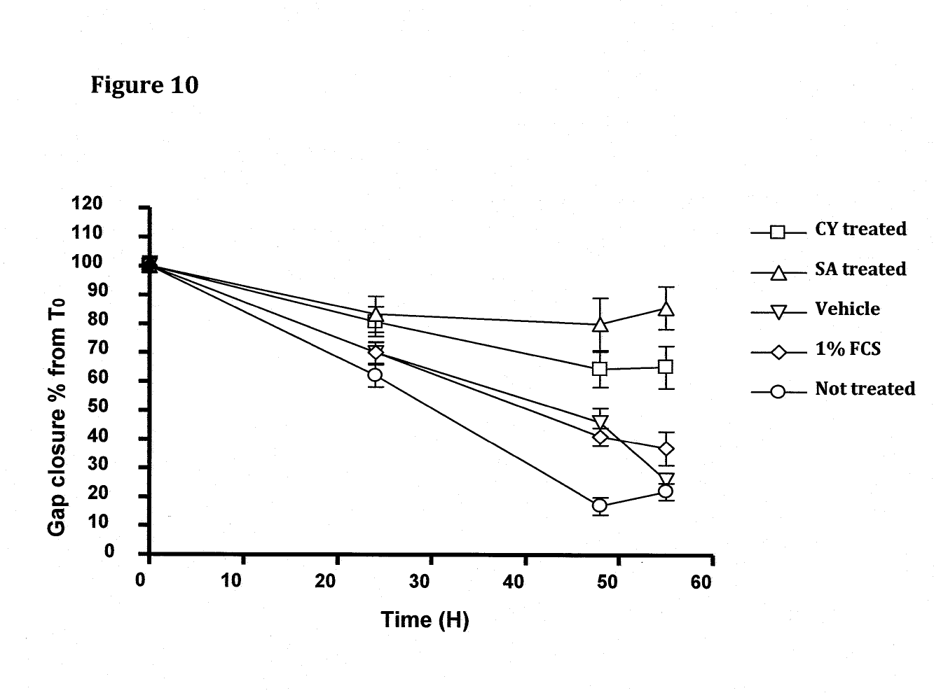

The results of the cytotoxicity assay are shown in Tables 2 and 3 and are graphically represented in FIG. 9. RPh-CY and RPh-SA showed similar effects, with both fractions being found to be substantially non-toxic to the cells at concentrations less than 2.25 mg/ml. At higher concentrations, RPh-SA exhibited slightly less toxic effects. The results of the scratch assay are provided in Tables 4 and 5, which, respectively, show gap width measurements in pixels and as a percentage of that at T0. FIG. 10 shows the results of data analysis involving the gap measurement at each time point.

TABLE 2

| | 1 | 2 | 3 | 4 | 5 | 6 | |
|---|---|---|---|---|---|---|---|
| A | SPL1:1 | SPL1:1 | SPL1:1 | SPL2:1 | SPL2:1 | SPL2:1 | Well ID |
| | 500 | 500 | 500 | 500 | 500 | 500 | Conc/Dil |
| | | | | | | | Name |
| B | SPL1:2 | SPL1:2 | SPL1:2 | SPL2:2 | SPL2:2 | SPL2:2 | Well ID |
| | 250 | 250 | 250 | 250 | 250 | 250 | Conc/Dil |
| | | | | | | | Name |
| C | SPL1:3 | SPL1:3 | SPL1:3 | SPL2:3 | SPL2:3 | SPL2:3 | Well ID |
| | 100 | 100 | 100 | 100 | 100 | 100 | Conc/Dil |
| | | | | | | | Name |
| D | SPL1:4 | SPL1:4 | SPL1:4 | SPL2:4 | SPL2:4 | SPL2:4 | Well ID |
| | 50 | 50 | 50 | 50 | 50 | 50 | Conc/Dil |
| | | | | | | | Name |
| E | SPL1:5 | SPL1:5 | SPL1:5 | SPL2:5 | SPL2:5 | SPL2:5 | Well ID |
| | 25 | 25 | 25 | 25 | 25 | 25 | Conc/Dil |
| | | | | | | | Name |
| F | SPL1:6 | SPL1:6 | SPL1:6 | SPL2:6 | SPL2:6 | SPL2:6 | Well ID |
| | 10 | 10 | 10 | 10 | 10 | 10 | Conc/Dil |
| | | | | | | | Name |
| G | SPLC1 | SPLC1 | SPLC1 | CTL1 | CTL1 | CTL1 | Well ID |
| | 0 | 0 | 0 | | | | Conc/Dil |
| | | | | | | | Name |
| H | BLK | BLK | BLK | SPL3 | SPL3 | SPL3 | Well ID |
| | | | | 10 | 10 | 10 | Conc/Dil |
| | | | | | | | Name |

SPL1—RphCY; SPL2—RphSA; SPLC1—Vehicle only; BLK—Blank (no cells); CTL1—Cells with medium - not treated; SPL3—DMSO 10%.

TABLE 3

| | 1 | 2 | 3 | 4 | 5 | 6 | |
|---|---|---|---|---|---|---|---|
| A | 0.288 | 0.27 | 0.269 | 0.31 | 0.305 | 0.307 | 450 |
| B | 0.292 | 0.294 | 0.316 | 0.321 | 0.315 | 0.313 | 450 |
| C | 1.661 | 1.502 | 1.327 | 1.323 | 1.281 | 1.21 | 450 |
| D | 1.582 | 1.493 | 1.414 | 1.377 | 1.309 | 1.295 | 450 |
| E | 1.627 | 1.483 | 1.512 | 1.515 | 1.491 | 1.418 | 450 |
| F | 1.549 | 1.486 | 1.481 | 1.474 | 1.498 | 1.462 | 450 |
| G | 1.582 | 1.493 | 1.452 | 1.449 | 1.425 | 1.361 | 450 |
| H | 0.289 | 0.305 | 0.295 | 0.238 | 0.233 | 0.234 | 450 |

TABLE 4
Gap width (pixels) at different time periods following treatment

| Time | | RPh—CY | RPh—SA | Vehicle | FCS | Untreated |
|---|---|---|---|---|---|---|
| 0 H | | 489 | 510 | 550 | 543 | 551 |
| | SD | 147 | 108 | 73 | 64 | 86 |
| 24 H | | 395 | 425 | 384 | 381 | 339 |
| | SD | 98 | 123 | 80 | 75 | 85 |
| 48 H | | 315 | 408 | 252 | 225 | 91 |
| | SD | 117 | 179 | 103 | 65 | 64 |
| 55 H | | 319 | 437 | 141 | 203 | 119 |
| | SD | 139 | 146 | 38 | 124 | 63 |

### Average gap T0=100%; 529±33.

TABLE 5
Gap width (% time 0 hr) at different
time periods following treatment

| Time | | | RPh—CY | RPh—SA | Vehicle | FCS | Untreated |
|---|---|---|---|---|---|---|---|
| 0 H | AVR | | 100 | 100 | 100 | 100 | 100 |
| | | SD | 30 | 21 | 13 | 12 | 16 |
| 24 H | AVR | | 81 | 83 | 70 | 70 | 62 |
| | | SD | 20 | 24 | 15 | 14 | 15 |
| 48 H | AVR | | 64 | 80 | 46 | 41 | 17 |
| | | SD | 24 | 35 | 19 | 12 | 12 |
| 55 H | AVR | | 65 | 86 | 26 | 37 | 22 |
| | | SD | 28 | 29 | 7 | 23 | 11 |

Single repeat n=15 measurements

The gap closure observed within each of the groups was consistent. The standard deviation obtained for each time point is mostly below 20% in the vehicle, FCS and untreated groups, and mostly below 30% in the RPh-CY and RPh-SA treated groups.

The results indicate that in untreated cells, and cells treated with vehicle or with FCS, gap closure of about 60%-80% occurred during the 55 hr incubation period, with most of the motility taking place within the first 48 hr.

In contrast, gap closure observed in treated cell cultures after 55 hr corresponded to up to 35% in RPh-CY treated cultures, and up to 14% in RPh-SA treated cultures, which was significantly lower compared to the untreated and vehicle treated groups. In addition, gap closure in the treated cell cultures was significantly lower than that observed in FCS-treated cells, indicating that the isolated Cupressaceae fractions were significantly more effective than the vehicle and the positive control in inhibiting fibroblast migration.

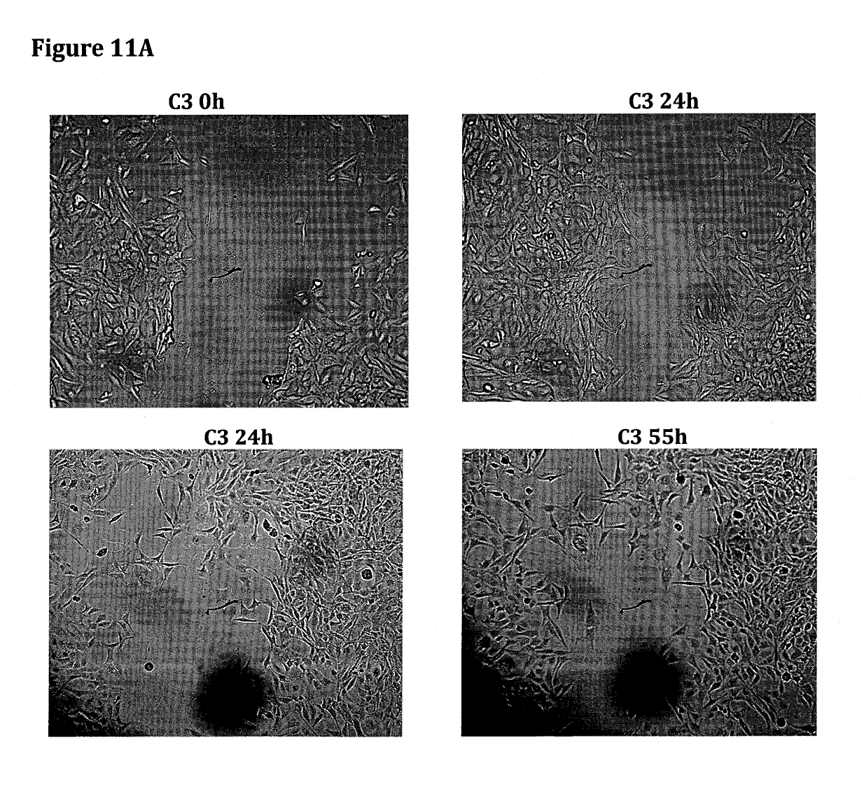

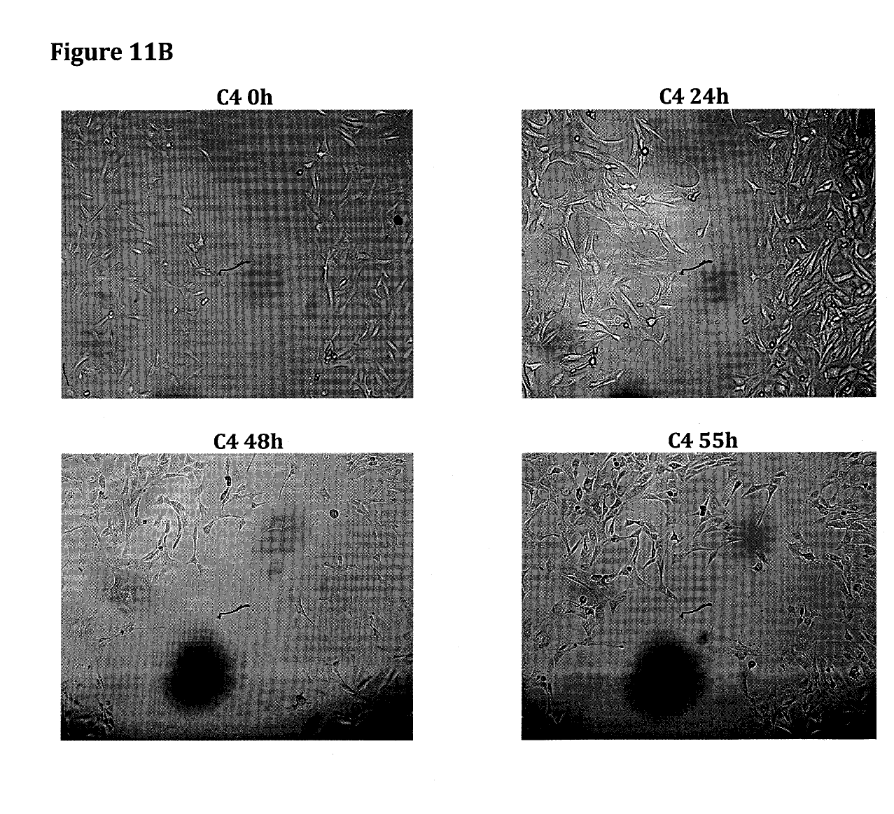

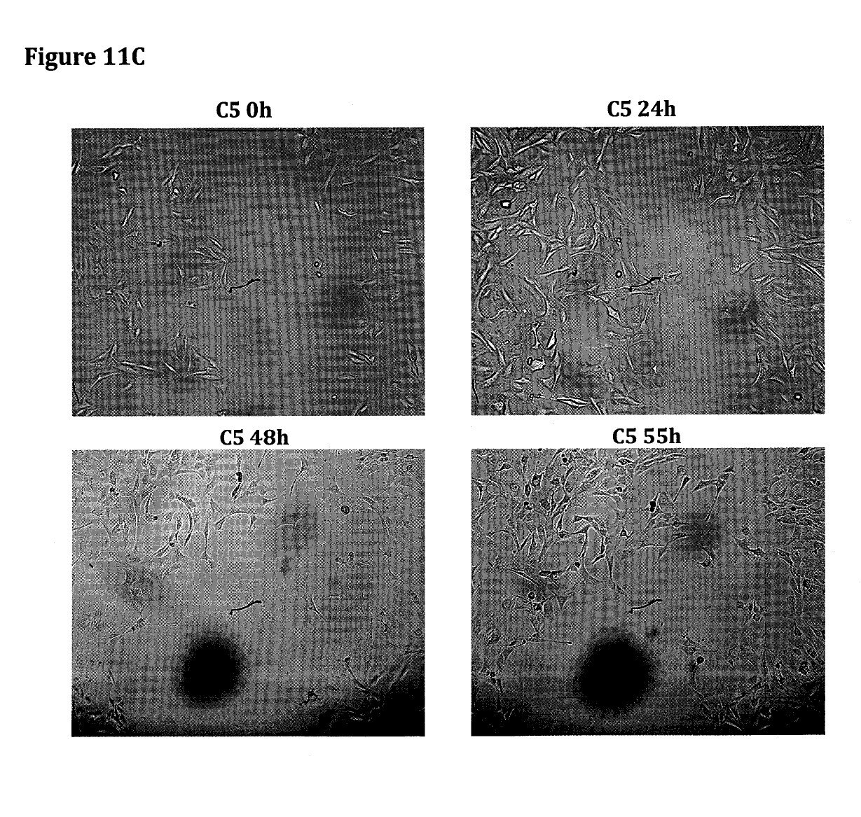

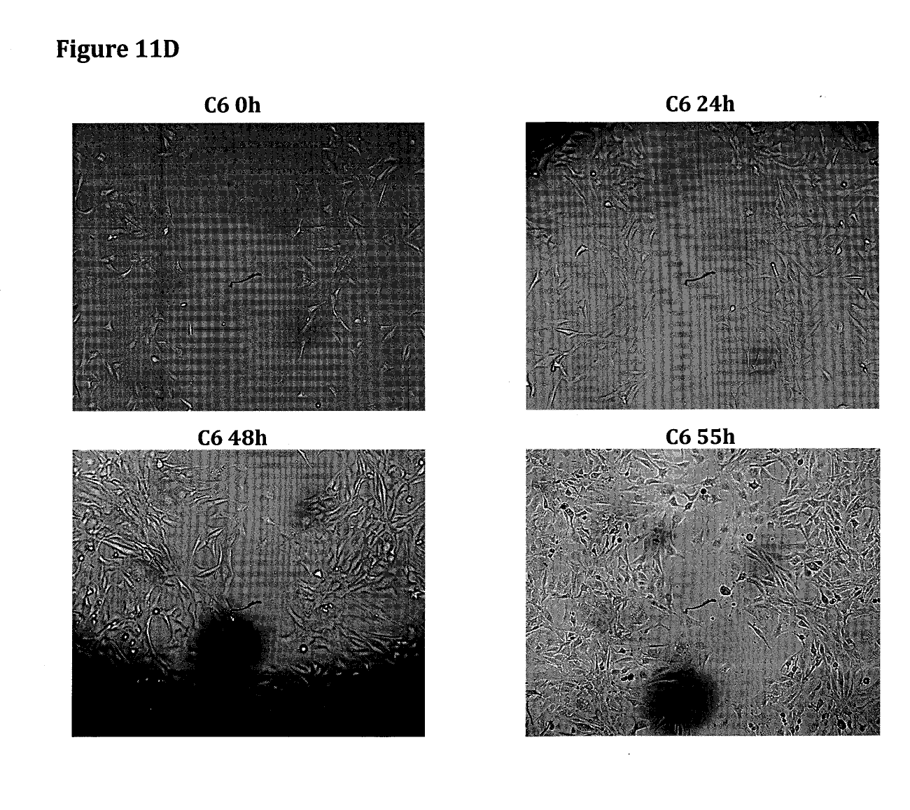

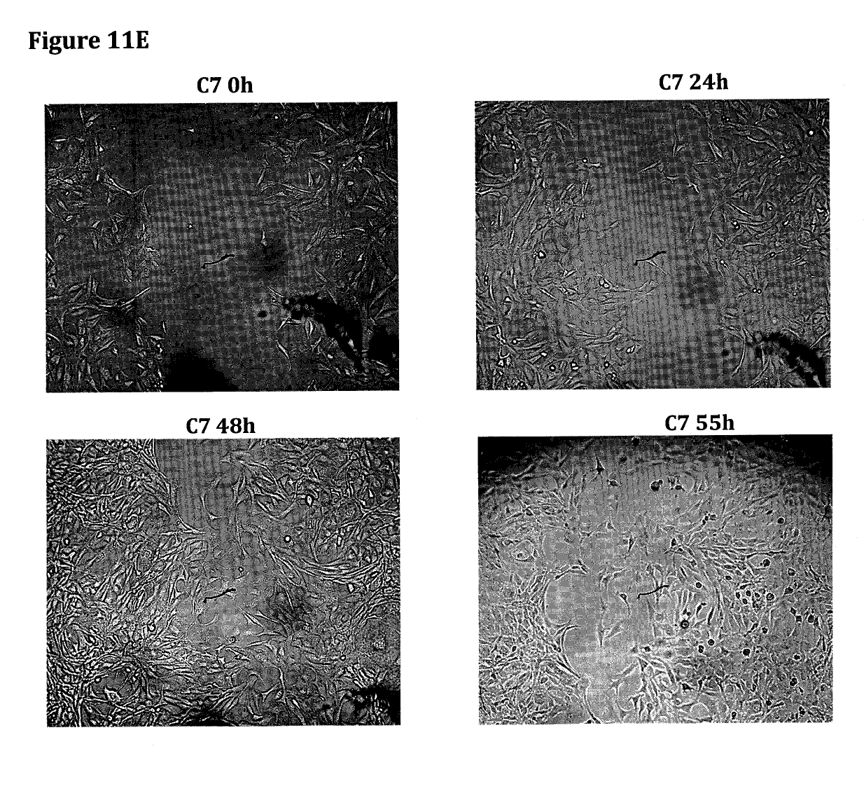

Visualization of the treated cultures (FIGS. 11A-E) indicate that RPh-SA induced breakdown of the monolayer at the treated dose so that within a few hours the intercellular contact was not visible and cells were separated but alive.

The test results reveal that RPh-CY and RPh-SA inhibit fibroblast proliferation and migration. Inhibition on the migratory capacity of fibroblasts indicates that RPh-CY and RPh-SA have potential to be used as therapeutic agents for high fibroblast cellularity seen in fibroproliferative disorders

### Example 12

### Preparation of Nanoemulsions of Isolated Fractions of Cupressaceae Resins

Liquid oil-in-water nanoemulsion formulations are prepared by high pressure emulsification techniques of all lipid ingredients and the active fraction dissolved in the lipid oil phase and emulsified with an aqueous phase, projected to result in the formation of stable, spheric and uniformly dispersed drug-containing lipid nanodroplets. The emulsion droplet size reduction is essential to generate drug formulations with high stability. Preferred nanoemulsion droplets have a mean droplet size of less than one micron (generally in the range of 0.1-0.2 μm) uniformly dispersed in an aqueous phase. The uniqueness of the large internal hydrophobic oil core of the nanoemulsion droplets provides high solubilization capacity for water insoluble compounds.

1. Preparation of Oil Phase

The oil phase is composed of 13% lipoid E-75, 0.026% αTP-succinate, propylparaben as antioxidant and 86.9% Miglyol® 810. A Cupressaceae resin extract prepared as in Example 1 or 3 is dissolved in the oil phase. The components are mixed with mild heating until a homogenous completely solubilized solution is obtained.

2. Preparation of Aqueous Phase

The aqueous phase is composed of 0.1% EDTA, 0.5% Tween-80, 2.3% glycerol, methylparaben as preservative and 97.1% water. pH was adjusted to 7.4 by NaOH 1N.

3. Mixing of Oil and Aqueous Phases

Oil phase (3.7 g) is heated and added to 70 ml of the aqueous phase (preheated). The mixture is gently stirred for 10-15 min at room temperature.

4. Preparation of Oil-in-Water Coarse Emulsion

An oil-in-water emulsion is prepared using the medium size dispenser and high shear homogenizing unit Polytron®, at 20,000 rpm for 5 min.

5. Sizing the Emulsion to Submicron Range by Gaulin® High Pressure Homogenizer

The droplet size of the emulsion obtained after step 4 is reduced to the submicron (nanosize) range by submitting the emulsion to high shear homogenization using the Gaulin® Microlab 70 high pressure homogenizer at 800 bar pressure. A total of 5-6 cycles should be performed to obtain homogenous nanoemulsion droplets having average particle size of less than 200 nm. Particle size is to be determined by photon correlation spectroscopy (PCS) using a N4MD particle size analyzer (Coulter® Electronics, UK). When most of the particles (>90%) are smaller than 200 nm, the sizing process is determined to be complete.

6. Sterile Filtration

Filtration at aseptic conditions of the nanoemulsion to sterile vials using a 0.2 μm PES sterile filter and storage at 40° C.

### Example 13

### Preparation of Spray-Dryed Powder of Isolated Fractions of Cupressaceae Resins

A convenient process for manufacturing the lipid mixture product of an isolated fraction of Cupressaceae resins is by direct spray-drying of the formulation from a mixture of non-polar solvent dispersion containing all the lipid ingredients and water containing the hydrophilic components, taking into account cost effectiveness and up-scaling considerations. The selected spray-drying method is optimized in order to get a fine, free-flowing powder. The isolated fraction of a Cupressaceae resin is dissolved in the lipid phase containing the lipid ingredients lecithin, tricaprin (capric acid triglyceride), tocopherol succinate and warmed (−40° C.) in a non-polar solvent until a good dispersion is obtained. A dispersion of fumed silicon dioxide (Cab-O-Sil®) in water (5%) was prepared by swelling the powder in purified water. The resultant slurry (prewarmed to 40° C.) is then poured slowly into the non-polar solvent lipid dispersion and the mixture is agitated at 40° C. for about 1 hr until a homogenous dispersion is obtained. The mixture is then spray-dried using the Yamato Pulvis® GA32 spray-dryer. The spray-drying conditions are: flow rate 7 ml/min, inlet temperature 130° C., outlet temperature 70° C., and drying air flow 0.5 m³/min. A homogeneous dry powder containing the isolated Cupressaceae fraction-lipid mixture is expected to be obtained.

The isolated Cupressaceae fraction-lipid mixture formulation prepared by the direct spray drying process is expected to show good water dispersibility, thus being suitable for the preparation of solid-dosage forms such as hard gelatin capsules or tablets for the enhanced oral delivery of the isolated cupressaceae fraction with potential good oral bioavailability.

### Example 14

### Preparation of Liposomal Preparations Containing Isolated Cupressaceae Fractions

Lipids containing dissolved isolated Cupressaceae fractions were dissolved in 100 ml dichloromethane in a round bottom flask, and stirred for 30 min at room temperature until a clear transparent solution was obtained. Solvent will be evaporated using a rotary evaporation unit at 39° C. First, the flask will be rotated at 4.5 rpm, 5 min under atmospheric pressure, followed by 10-30 min (until full evaporation of the solvent) under weak vacuum, and finally 15 min under full vacuum. At the end of the evaporation process a uniform lipid film will be created. The lipid film will be dissolved in 15 ml isotonic buffer. Liposomes are prepared by vigorous shaking for 10-30 min using multi-wrist shaker, until a uniform and milky dispersion of multilamellar vehicle (MLV) will be formed and no remaining lipid film will be apparent. In order to obtain an equilibrated and homogenous liposome preparation the flask will be further shaken at 37° C. for 30-90 min. at 270 rpm.

### Example 15

### Preparation of Microemulsions Containing Isolated Fractions of Cupressaceae Resins

Several surfactants commonly used in parenterals may be utilized to develop water-in-oil and oil-in-water-microemulsions acceptable for injectable, oral and topical use. The pharmaceutically acceptable surfactants suitable for the formation of microemulsion formulations are non-ionic surfactants including polyoxyl 40 hydrogenated castor oil (sold under the trade name Cremophor RH40®), polyoxyl 35 castor oil (sold under the trade name Cremophor® EL), polyoxyethylene sorbitan fatty acid esters (polysorbates), poloxamers (Pluronics®), vitamin E-TPGS 1,000 (VE-TPGS 1,000), polyoxyethylene alkyl ethers, Solutol® HS-15, Tagat® TO, Peglicol 6-oleate, polyoxyethylene sterates, or saturated polyglycolyzed glycerides, all of which are commercially available. The preferred surfactants include polyoxyl 40 hydrogenated castor oil (Cremophor® RH40®), polyoxyl 35 hydrogenated castor oil (Cremophor® EL), polyoxyethylene sorbitan fatty acid esters (polysorbates), poloxamers (Pluronics®), and vitamin E-TPGS 1,000. The total amount of the surfactant present in the composition will be generally from about 100 to about 700 mg/g, and preferably from about 300 to about 500 mg/g.

Preparation of microemulsions containing isolated fractions of Cupressaceae resins may be performed by dissolving the isolated fractions in an appropriate amount of oil such as medium chain tryglycerides (Miglyol) in a suitable vial. The vial is then capped. The vial is put into a water bath of about 50-60° C. and shaken gently until all of the drug material is completely dissolved. After the vial is cooled to room temperature, an appropriate amount of surfactant (such as Cremophor® EL or VE-TPGS) is added and followed by the mixture of mono- and di-glycerides of fatty acids, if any. The vial is then capped and placed into the water bath of about 50-60° C. The vial is shaken gently to obtain a clear, uniform solution. This solution can be filled into HPMC capsules and stored at room temperature before oral dosing. Alternatively, the substituted polymer powders (such as HPMC) can be added into the solution with adequate agitation (i.e., stirring, shaking) to obtain a uniform polymer suspension. The resulting composition can then be filled into either soft gelatin or hard gelatin capsules and stored at room temperature before oral dosing. Alternatively the microemulsion formulation can be used as a topically or filtered through 0.2 um membranes to be administered parenterally.

The microemulsions containing isolated Cupressaceae fractions are expected to have good water-dispersibility properties and self-emulsify when diluted in aqueous media to form small nanometric micelles that with enhanced bioavailability.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. An isolated fraction of a Cupressaceae resin, wherein the fraction is characterized in that it is soluble in at least one polar organic solvent and soluble in at least one non-polar organic solvent, and wherein said fraction is substantially devoid of compounds which are soluble in said polar organic solvent but insoluble in said non-polar organic solvent.

2. The isolated fraction according to claim 1, comprising at least one compound selected from the group consisting of a monoterpene, a sesquiterpene, a diterpene, a triterpene, a C15-tropolone, a sesquiterpenoid, a diterpenoid and a triterpenoid and combinations thereof.

3. The isolated fraction according to claim 1, comprising at least one compound selected from the group consisting of sempervirol, totarol, ferruginol, manool, torusolol, torusolal, isoagatholal, agathadiol, nootkatin, chanootin, sandaracopimaric acid, sandaracopimarol, 4-epidehydroabietic acid, communic acid and combinations thereof.

4. (canceled)

5. The isolated fraction according to claim 1, wherein the isolated fraction is substantially devoid of terpene compounds which are soluble in said polar organic solvent and insoluble in said non-polar organic solvent; or wherein the isolated fraction is substantially devoid of terpenoid compounds which are soluble in said polar organic solvent and insoluble in said non-polar organic solvent.

6. The isolated fraction according to claim 1, wherein the Cupressaceae plant material is from a species is selected from the group consisting of Tetraclinis articulata, Cupressus sempervirens and Juniperus communis.

7-8. (canceled)

9. The isolated fraction according to claim 1, having an HPLC chromatogram substantially as depicted in FIG. 1A or FIG. 1B, or having an HPLC chromatogram substantially as depicted in FIG. 4.

10. (canceled)

11. The isolated fraction according to claim 1, obtained by a process comprising:
(a) treating a Cupressaceae resin with a polar organic solvent;
(b) isolating a fraction soluble in said polar organic solvent;
(c) optionally removing said polar organic solvent;
(d) treating the soluble fraction obtained in step (b) or (c) with a non-polar organic solvent,
(e) isolating a fraction soluble in said non-polar organic solvent; and
(f) optionally removing said non-polar organic solvent;
wherein steps (d) to (f) may precede steps (a) to (c).

12. The isolated fraction according to claim 11, wherein the polar solvent is selected from the group consisting of an alcohol, an ether, an ester, an amide, an aldehyde, a ketone, a nitrile, and combinations thereof; and wherein the non-polar organic solvent is selected from the group consisting of acyclic or cyclic, saturated or unsaturated aliphatic hydrocarbons and aromatic hydrocarbons, each of which is optionally substituted by one or more halogens, and combinations thereof.

13-22. (canceled)

23. A pharmaceutical composition comprising an effective amount of the isolated fraction according to claim 1, and a pharmaceutically acceptable carrier.

24-27. (canceled)

28. The pharmaceutical composition according to claim 23, wherein the carrier is selected from the group consisting of at least one oil, at least one wax and combinations thereof.

29. The pharmaceutical composition according to claim 28, wherein the at least one oil is selected from the group consisting of almond oil, canola oil, coconut oil, corn oil, cottonseed oil, grape seed oil, olive oil peanut oil, saffron oil, sesame oil, soybean oil and combinations thereof.

30. The pharmaceutical composition according to claim 23, comprising from about 0.01 to about 25% (w/w) of an isolated fraction of Cupressaceae resin, based on the total weight of the composition.

31. The pharmaceutical composition according to claim 23, in a form suitable for administration by a route selected from the group consisting of oral, topical, parenteral, intramuscular, subcutaneous, intradermal, vaginal, rectal, intracranial, intranasal, intraocular, auricular, pulmonary intralesional, intraperitoneal, intraarterial, intracerebral, intracerebroventricular, intraosseus and intrathecal.

32. The pharmaceutical composition according to claim 23, in a form suitable for administration by injection.

33. The pharmaceutical composition according to claim 23, in a form selected from the group consisting of a capsule, a tablet, a suppository, a suspension, an ointment, a cream, a lotion, a solution, an emulsion, a film, a cement, a powder, a glue, an aerosol and a spray.

34-36. (canceled)

37. A method of treating impaired neurological function, the method comprising administering an effective amount of the pharmaceutical composition of claim 23 to a subject in need thereof, thereby treating impaired neurological function.

38. A method of preventing or treating a fibrotic condition, the method comprising administering an effective amount of the pharmaceutical composition of claim 23 to a subject in need thereof, thereby treating the fibrotic condition.

39. A method of preventing or reducing scar formation at a wound site, the method comprising administering to a wound site in a subject in need thereof an effective amount of the pharmaceutical composition of claim 23, thereby preventing or reducing scar formation at a wound site.

40. The method of claim 37, wherein the impaired neurological function is associated with a condition or disease selected from the group consisting of Alzheimer's disease, amyotrophic lateral sclerosis (ALS), multiple sclerosis, Parkinson's disease, vascular dementia and senile dementia.

41. The method of claim 40, wherein the impaired neurological function is associated with Alzheimer's disease.

42. The method of claim 37, wherein the impaired neurological function is associated with trauma or stroke.

43. The method of claim 38, wherein the fibrotic condition is selected from the group consisting of arterial fibrosis, arthrofibrosis, bladder fibrosis, breast fibrosis, cardiac fibrosis, endomyocardial fibrosis, liver fibrosis, lymph node fibrosis, mediastinal fibrosis, muscle fibrosis, myelofibrosis, nephrogenic systemic fibrosis, pancreatic fibrosis, pleural fibrosis progressive massive fibrosis, pulmonary fibrosis, renal fibrosis, retroperitoneal fibrosis, skin fibrosis, thyroid fibrosis, cirrhosis, vascular stenosis, restenosis, and chronic obstructive pulmonary disease (COPD).

44. The method of claim 38, wherein the fibrotic condition is selected from the group consisting of scleroderma, a fibromatosis and hypertrophic scarring.

45. The method of claim 39, for preventing or reducing scar formation at a wound site.

46. The isolated fraction according to claim 45, wherein the wound site comprises a wound selected from the group consisting of a burn, an amputation wound, a split-skin donor graft, a skin graft donor site, a medical device implantation site, a bite wound, a frostbite wound, a puncture wound, a shrapnel wound and a surgical wound.
